(19) United States
(12) Patent Application Publication
(10) Pub. No.: US 2026/0235582 A1
(43) Pub. Date: Aug. 13, 2026

(54) CHRONIC GRAFT-VERSUS-HOST DISEASE (CGVHD) DIAGNOSTIC AND RISK BIOMARKER PANELS, METHODS AND USES THEREOF

(71) Applicants: THE UNIVERSITY OF BRITISH COLUMBIA, Vancouver (CA); THE UNIVERSITY OF MANITOBA, Winnipeg (CA)

(72) Inventors: Kirk R. Schultz, Vancouver (CA); Bernard Ng, Vancouver (CA); Geoff D. E. Cuvelier, Winnipeg (CA)

(21) Appl. No.: 19/105,976

(22) PCT Filed: Aug. 30, 2023
(86) PCT No.: PCT/CA2023/051152

§ 371 Date: Feb. 24, 2025

Related U.S. Application Data
(60) Provisional application No. 63/402,943, filed on Aug. 31, 2022.

(51) Int. Cl. G01N 33/50; G01N 33/68; G16H 50/30

(52) U.S. Cl. CPC G01N 33/5091; G01N 33/6893; G16H 50/30; G01N 2333/522; G01N 2333/70525; G01N 2333/96494; G01N 2800/50

(57) ABSTRACT

This invention provides chronic graft-versus-host disease (cGVHD) biomarker panels for use in diagnostic and risk assignment methods. More particularly, the invention relates to particular combinations of biomarkers for diagnosing cGVHD and for predicting whether there is a low, intermediate or high risk of developing cGVHD. Furthermore, there are provided different treatment options depending on a patient's risk level or whether they have moderate to severe cGVHD.

### CROSS REFERENCE TO RELATED APPLICATIONS

This application claims the benefit of U.S. Provisional Patent Application Ser. No. 63/402,943 filed 31 Aug. 2022 entitled “CHRONIC GRAFT-VERSUS-HOST DISEASE (CGVHD) DIAGNOSTIC AND RISK BIOMARKER PANELS, METHODS AND USES THEREOF”.

### FIELD OF THE INVENTION

This invention relates to chronic graft-versus-host disease (cGVHD) and biomarker panels and their use in diagnostic and risk assignment methods, with optional subsequent treatments. More particularly, the invention relates to particular combinations of biomarkers for diagnosing cGVHD and for predicting whether there is a low, intermediate or high risk of developing cGVHD. Furthermore, there are provided different treatment options depending on a patient's risk level or whether they have moderate to severe cGVHD, whether they have pulmonary cGVHD, de novo cGVHD, or progressive cGVHD.

### BACKGROUND

Allogeneic hematopoietic stem cell transplant (HSCT) is performed as part of the management of high-risk leukemias and several non-malignant disorders in children and adults. The hematopoietic cells are usually derived from bone marrow, peripheral blood, or umbilical cord blood. For HSCT patients, cGVHD remains a major long-term complication, negatively impacting upon quality of life while increasing morbidity and mortality.¹⁸ Historically, pediatric cGVHD has been an understudied disease due to the reduced incidence of cGVHD in children relative to adults, and the inherent difficulty in researching a rare disorder using a multi-institutional study design.

cGVHD has a complex disease pathogenesis. Multiple arms of the innate and adaptive immune systems operate in parallel to produce an alloreactive disease characterized by tissue injury, chronic inflammation, dysregulated immunity, aberrant tissue repair and fibrosis.^{9,10} clinical manifestations of cGVHD are variable between patients, including timing of onset, organs affected, severity, and natural history. To address this, the 2005 and 2014 National Institutes of Health Consensus Criteria (NIH-CC) were created to impart minimal diagnostic criteria for cGVHD.^{11,12} Despite these criteria, clinicians still experience challenges in diagnosing cGVHD, particularly early in its onset (when signs and symptoms are in development and less specific) or when the clinician is faced with an atypical manifestation of the disease.¹³ Even experienced pediatric transplant physicians misclassify cGVHD in 28% of cases, with manifestations initially thought related to cGVHD being better classified as due to either late-acute GVHD (L-aGVHD) or an alternative non-GVHD diagnosis (e.g., infections, drug-reactions) following central study adjudication.¹⁴ Clinically useful diagnostic biomarkers could therefore greatly aid clinicians in the diagnosis of cGVHD, particularly at its onset and early stages of development.¹⁵ To date, no cGVHD biomarkers are validated or available for routine clinical use.¹⁶

The Applied Biomarkers of Late Effects of Childhood Cancer (ABLE)/Pediatric Blood and Marrow Transplant Consortium (PBMTC) 1202 study (NCT:02067832) evaluated diagnostic and risk assignment biomarkers before and at the onset of pediatric cGVHD using a prospective study design at 27 HSCT centers.^{14,17,18} Using clinical cohorts of pediatric patients who had undergone extensive clinical adjudication of their GVHD status,¹⁴ we analyzed several individual cellular and plasma markers in patients with and without cGVHD. Given the heterogeneous nature of cGVHD, we further developed machine learning-based models that combine multiple clinical factors with cellular and plasma biomarkers for diagnosing pediatric cGVHD and making pediatric cGVHD risk assignments.

### SUMMARY

This invention is based in part on the discovery that particular panels of biomarkers are diagnostic of cGVHD, and a different panel of biomarkers measured at day 100 post HSCT that is useful for assigning risk of whether a patient will develop cGVHD in the future. Fortuitously, particular combinations of these markers are suitable for use in cGVHD diagnostic methods and cGVHD risk assignment methods, respectively. The invention also provides for alternative treatment methods depending on the risk assignment or whether or not cGVHD is diagnosed.

As shown herein, we were able to: (1) define diagnostic biomarkers at the onset of cGVHD in children that would complement the NIH-CC; (2) develop a diagnostic classifier that could help clinicians differentiate cGVHD at its onset from other non-cGVHD manifestations; (3) determine the risk of developing cGVHD based on early biomarker measurements at about 100 days after HSCT; and (4) provide clinically applicable diagnostic biomarkers of pediatric cGVHD and subsequent treatment decisions. To our knowledge, this is the largest cohort of pediatric patients with cGVHD reported in a prospective, multi-institution study design. However, given the similarities of NK_{REGS} (decreases) and cytokine (increases—i.e. ST2) between adults and children in cGVHD patients⁴⁰, it is expected that the results disclosed herein will also be applicable to adult cGVHD patients.

The National Institutes of Health Consensus Criteria for cGVHD diagnosis can be challenging to apply in children, making pediatric cGVHD diagnosis difficult. We aimed to discover diagnostic pediatric cGVHD biomarkers that would complement current clinical criteria and help differentiate cGVHD from non-cGVHD diagnoses. The Applied Biomarkers of Late Effects of Childhood Cancer (ABLE) study, open at 27 transplant centers, prospectively evaluated 302 pediatric patients after hematopoietic cell transplant (234 evaluable). Forty-four patients developed cGVHD. Mixed and fixed effect regression analyses were performed on diagnostic cGVHD onset blood samples for cellular and plasma biomarkers, with individual markers declared relevant if they met three criteria: an effect ratio≥1.3 or ≤0.75; an area under the curve (AUC) of ≥0.60; and a p-value<5.814×10-4 (Bonferroni correction) (mixed effect) or <0.05 (fixed effect). To address the complexity of cGVHD diagnosis in children, we further built a machine learning-based classifier that combined multiple cellular and plasma biomarkers with clinical factors. Decreases in NK_{REGS}, naïve CD4 helper T cells, and naïve regulatory T cells; and elevations in CXCL9, CXCL10, CXCL11, ST2, ICAM-1, and sCD13 characterized the onset of cGVHD. Evaluation of time-dependence revealed that sCD13, ST2, and ICAM-1 varied with timing of cGVHD onset. The cGVHD diagnostic classifier achieved an AUC of 0.89 with a positive predictive value of 82% and negative predictive value of 80% for diagnosing cGVHD. We also built a machine learning-based risk assignment algorithm for predicting whether a patient will develop cGVHD in the future based on biomarker measurements taken at 100 days after HSCT. The cGVHD risk assignment algorithm achieved at AUC of 0.78 with a positive predictive value of 74% and a negative predictive value of 68%.

In a first aspect, there is provided a chronic graft-versus-host disease (cGVHD) diagnostic biomarker panel, the panel including: (a) a NK_{REG} Non-Cytolytic cell that may be selected from one or more of: CD56^{Bright} Perforin^{Negative} CD56; and CD56^{Bright} Granzyme B^{Negative} CD56; (b) a Naïve Helper T cell that may be selected from one or more of: CCR7+ CD45RA+ CD4+; PD1− CD45RA+ CD4+; CD27+ CD45RA+ CD4+; and CD31+ CD45RA+ CD4+; (c) a Naïve Cytotoxic T Cell that may be CCR7+ CD45RA+ CD8+; (d) a Naïve Regulatory T Cell that may be selected from one or more of: PD1− CD45RA+ Treg; and CD31+ CD45RA+ Treg; (e) a chemokine that may be selected from one or more of: CXCL9; CXCL10; and CXCL11; (f) an intercellular adhesion molecule-1 (ICAM-1); (g) a T-cell immunoglobulin and mucin domain-3 (TIM-3); and (h) a suppression of tumorigenicity 2 (ST2).

The cGVHD diagnostic biomarker panel may include: (a) a NK_{REG} Non-Cytolytic cell that may be selected from one or more of: CD56^{Bright} CD355⁺ CD56; CD56^{Bright} Perforin^{Negative} CD56; and CD56^{Bright} Granzyme B^{Negative} CD56; (b) a Cytolytic N K cell that may be CD56^{Lo} Granzyme B^{Hi} CD56; (c) a Naïve Helper T cell that may be selected from one or more of: CD4+ CD45RA+ CD3; CCR7+ CD45RA+ CD4+; PD1− CD45RA+ CD4+; CD27+ CD45RA+ CD4+; and CD31+ CD45RA+ CD4+; (d) a Memory T Helper Cell that may be CCR7− CD45RA− CD4+; (e) a Naïve Cytotoxic T Cell that may be selected from one or more of: CCR7+ CD45RA+ CD8+; and CD27+ CD45RA+ CD8+; (f) a Naïve Regulatory T Cell that may be selected from one or more of: PD1− CD45RA+ Treg; and CD31+ CD45RA+ Treg; (g) a chemokine that may be selected from one or more of: CXCL9; CXCL10; and CXCL11; (h) an intercellular adhesion molecule-1 (ICAM-1); (i) a T-cell immunoglobulin and mucin domain-3 (TIM-3); (j) a suppression of tumorigenicity 2 (ST2); and (k) a matrix metalloproteinase-3 (MMP-3).

The cGVHD diagnostic biomarker panel may further include one or more clinical factors selected from: (i) malignant; or non-malignant disease; (ii) recipient age; (iii) graft type (PBSC, bone marrow, or umbilical cord); (iv) donor gender; and recipient gender; (v) HLA match; or HLA mismatch; (vi) ABO match; or ABO mismatch; (vii) sibling donor source; or unrelated donor source; (viii) myeloablative; or reduced-intensity conditioning; (ix) use of serotherapy; (x) use of total body irradiation; and (xi) days post-HSCT.

In a further aspect, there is provided a diagnostic method of detecting the expression level of a biomarker panel in a patient receiving a hematopoietic stem cell transplantation (HSCT), the diagnostic method including: (A) measuring, in a biological sample obtained from the patient, a relative cell % or a concentration of at least one of each type of biomarker in a diagnostic biomarker panel, wherein the diagnostic biomarker panel is set out herein; and (B) assigning a diagnosis of cGVHD or late acute GvHD.

The diagnostic panel may include: (a) a NK_{REG} Non-Cytolytic cell that may be selected from one or more of: CD56^{Bright} Perforin^{Negative} CD56; and CD56^{Bright} Granzyme B^{Negative} CD56; (b) a Naïve Helper T cell that may be selected from one or more of: CCR7+ CD45RA+ CD4+; PD1− CD45RA+ CD4+; CD27+ CD45RA+ CD4+; and CD31+ CD45RA+ CD4+; (c) a Naïve Cytotoxic T Cell that may be CCR7+ CD45RA+ CD8+; (d) a Naïve Regulatory T Cell that may be selected from one or more of: PD1− CD45RA+ Treg; and CD45RA+ Treg; (e) a chemokine that may be selected from one or more of: CXCL9; CXCL10; and CXCL11; (f an intercellular adhesion molecule-1 (ICAM-1); (g) a T-cell immunoglobulin and mucin domain-3 (TIM-3); and (h) a suppression of tumorigenicity 2 (ST2).

The patient that may be assigned a diagnosis of cGVHD where the biological sample has: (a) decreased CD56^{Bright} CD355⁺ CD56; CD56^{Bright} Perforin^{Negative} CD56; or CD56^{Bright} Granzyme B^{Negative} CD56 as a % of total lymphocytes; (b) increased CD56^{lo} G⁺ CD56 as a % of total lymphocytes; (c) decreased CD4+CD45RA+ naïve helper T cells as a % of CD4+ that may be selected from one or more of: CD4+CD45RA+CD3; CCR7+ CD45RA+ CD4+; PD1− CD45RA+ CD4+; CD27+CD45RA+CD4+; and CD31+CD45RA+CD4+; (d) increased CCR7-ve CD45RA-ve CD4+ effector memory helper T cells as a % of CD4+ cells; (e) decreased Naïve Cytotoxic T Cell that may be selected from one or more of: CCR7⁺ CD45RA⁺ CD8; or CD27⁺ CD45RA⁺ CD8; (f) decreased naïve regulatory T cells as a % of total regulatory T cells, that may be selected from CD45RA+PD1-ve and CD45RA+CD31+; (g) increased concentration of one or more of: CXCL9; CXCL10; and CXCL11; (h) increased concentration of ICAM-1; (i) increased concentration of ST2; (j) increased concentration of TIM-3; and (k) increased concentration of MMP3; or (1) decreased CD56^{Bright} Perforin^{Negative} CD56; or CD56^{Bright} Granzyme B^{Negative} CD56 as a % of total lymphocytes; (m) decreased CD4+CD45RA+ naïve helper T cells as a % of CD4+ that may be selected from one or more of CCR7+ CD45RA+CD4+; PD1− CD45RA+ CD4+; CD27+ CD45RA+ CD4+; and CD31+ CD45RA+ CD4+; (n) decreased Naïve Cytotoxic T Cell that may be CCR7⁺ CD45RA⁺ CD8; (o) decreased naïve regulatory T cells as a % of total regulatory T cells, that may be selected from CD45RA+PD1-ve and CD45RA+CD31+; (p) increased concentration of one or more of: CXCL9; CXCL10; and CXCL11; (q) increased concentration of ICAM-1; (r) increased concentration of ST2; and (s) increased concentration of TIM-3; as compared to a control non-cGVHD or to a non-cGVHD standard at an equivalent time period post HSCT.

The patient may be assigned a diagnosis of early-cGVHD, mid-cGVHD or late-cGVHD where the diagnostic markers were present at less than 4 months for early-cGVHD; 4-8 months for mid-cGVHD; and greater than 8 months for late-cGVHD.

The patient may be assigned a diagnosis of: (a) early-cGVHD where the biological sample has: (i) an increased concentration of one or more of: CXCL9; CXCL10; and CXCL11; (ii) an increased concentration of ICAM-1; and (iii) an increased enzyme activity of sCD13; (b) mid-cGVHD where the biological sample has: (i) an increased concentration of one or more of: CXCL9; CXCL10; and CXCL11; and (ii) an increased concentration of ST2; or (c) late-cGVHD where the biological sample has: (i) an increased concentration of one or more of: CXCL9; CXCL10; and CXCL11; (ii) an increased concentration of ICAM-1; (iii) an increased enzyme activity of sCD13; and (iv) an increased concentration of ST2; as compared to a control non-cGVHD or to a non-cGVHD standard at an equivalent time period post HSCT.

The patient may be assigned a diagnosis of moderate to severe cGVHD in the first year post-HSCT, where the biomarkers were as follows: (a) decreased CD56^{Bright} CD3^{Negative} Granzyme B^{Negative} CD56 as a % of total lymphocytes; (b) non-cytolytic NK cells no longer met criteria; (c) increased concentration of one or more of: CXCL9; CXCL10; and CXCL11; (d) increased concentration of ST2; (e) increased concentration of ICAM-1; and (f) increased enzyme activity of sCD13; as compared to a control non-cGVHD or to a non-cGVHD standard at an equivalent time period post HSCT.

The biological sample may be obtained from a pediatric patient. The biological sample may be obtained from an adult patient.

The biological sample may be obtained from the patient at: +100 days from HSCT (±14 days); 6 months from HSCT (±1 month); and 12 months from HSCT (±1 month). The biological sample may be selected from the group consisting of: whole blood; plasma; and tissue. The detecting of the expression level of a biomarker may be via one or more of the following assay methods: flow cytometry; microarray analysis; immunoassay; immunohistochemistry; enzymatic assay; and mass spectrometry. The immune assay may be selected from: enzyme-linked immunoassay (ELISA); and meso scale.

The cGVHD patient may be preferentially administered an immunosuppressive therapy (IST) selected from one or more of: sirolimus; mycophenolate mofetil; mycophenolate sodium; ibrutinib; ruxolitinib; belomosudil; thalidomide; azathioprine; pentostatin; daclizumab*; infliximab*; and rituximab*; or a biosimilar thereof*. The cGVHD patient may be further administered a steroid treatment. The cGVHD patient may be alternatively administered a steroid treatment.

In a further aspect, there is provided a cGVHD risk assignment biomarker panel, the panel including: (a) a CD19+ lymphocyte; (b) a transitional B cell selected from one or more of: a CD38− CD10− CD19+ B cell; a CD38^{dim} CD10^{lo} CD19+ B cell; and a CD38^{int} CD10^{int} CD19+ B cell; (c) an NK_{REG} Non-Cytolytic cell selected from one or more of: a CD56^{hi} CD355^{hi} CD56; a CD56^{hi} Perforin^{Negative} CD56; and a CD56^{hi} Granzyme B^{Negative} CD56; (d) a Cytolytic NK cell selected from one or more of: a CD56^{dim} P^{hi} CD56; a CD56^{dim} CD69+CD56+; and a CD56^{lo} G^{hi} CD56; (e) a CD3+ lymphocyte; (f) a PD1++CD45RA+ CD4 Follicular T cell; (g) a PD1+CD45RA+ CD8 T cell; (h) a CD31− CD45RA+ CD4 T cell; (i) an IgD+ CD27− CD19+B cell; (j) a lysophosphatidylcholine (LYSOC) selected from one or more of: LYSOC14:0; LYSOC16:1; and LYSOC20:3; (k) a Taurine; (l) an Aspartic acid; (m) a Phenylalanine; (n) an alpha-Ketoglutaric acid; (o) a Fumaric acid; and (p) a phosphatidylcholine (PC) PC40:6AA.

The cGVHD risk assignment biomarker panel may include: (a) a CD19+ lymphocyte; (b) a transitional B cell that may be selected from one or more of: a CD38− CD10− CD19+ B cell; a CD38^{dim} CD10^{lo} CD19+ B cell; and a CD38^{int} CD10^{int} CD19+ B cell; (c) an NK_{REG} Non-Cytolytic cell that may be selected from one or more of: a CD56^{hi} CD355^{hi} CD56; a CD56^{hi} Perforin^{Negative} CD56; and a CD56^{hi} Granzyme B^{Negative} CD56; (d) a Cytolytic NK cell that may be selected from one or more of: a CD56^{dim} P^{hi} CD56; a CD56^{dim} CD69+CD56+; and a CD56^{lo} G^{hi} CD56; (e) a CD3+ lymphocyte; (f) a PD1++CD45RA+ CD4 Follicular T cell; (g) a PD1+CD45RA+ CD8 T cell; (h) a CD31− CD45RA+ CD4 T cell; (i) a lysophosphatidylcholine (LYSOC) that may be selected from one or more of: LYSOC16:1; and LYSOC20:3; (j) a Taurine; (k) an Aspartic acid; (l) a Phenylalanine; (m) an alpha-Ketoglutaric acid; (n) a Fumaric acid; and (o) a phosphatidylcholine (PC) PC40:6AA.

The cGVHD risk assignment biomarker panel may further include one or more clinical factors selected from: (i) malignant; or non-malignant disease; (ii) recipient age; (iii) graft type (PBSC, bone marrow, or umbilical cord); (iv) donor gender; and recipient gender; (v) HLA match; or HLA mismatch; (vi) ABO match; or ABO mismatch; (vii) sibling donor source; or unrelated donor source; (viii) myeloablative; or reduced-intensity conditioning; (ix) use of serotherapy; (x) use of total body irradiation; and (xi) days post-HSCT.

In a further aspect, there is provided a risk assignment method of detecting the expression level of a biomarker panel in a patient about to receive a HSCT, the risk assignment method including: (A) measuring, in a biological sample obtained from the patient, a relative cell % or a concentration of at least one of each type of biomarker in a risk assignment biomarker panel, wherein the risk assignment biomarker panel is set out in claim 17 or 18; and (B) assigning a risk to the patient of developing cGVHD of either low, moderate or high; as compared to a control non-cGVHD or to a non-cGVHD standard at an equivalent time period post HSCT.

The patient may be assigned a diagnosis of cGVHD where the biological sample has: (a) decreased CD56^{Bright} CD355⁺ CD56; CD56^{Bright} Perforin^{Negative} CD56; or CD56^{Bright} Granzyme B^{Negative} CD56 as a % of total lymphocytes; (b) increased CD56^{lo} P⁺ CD56; CD56^{lo} CD69⁺ CD56; or CD56^{lo} G⁺ CD56 as a % of total lymphocytes; (c) increased CD3+ T cells as a % of total lymphocytes; (d) increased PD1++ CD45RA+CD4+ T cells as a % of total lymphocytes; (e) decreased CD31− CD45RA+ CD4+ as a % of CD4+ cells; (f increased PD1+ CD45RA+CD8+ T cells as a % of total lymphocytes; (g) decreased CD19+lymphocyte cells as a % of lymphocyte cells; (h) decreased transitional B cell selected from one or more of: a CD38− CD10− CD19+ B cell; a CD38^{dim} CD10^{lo} CD19+ B cell; and a CD38^{int} CD10^{int} CD19+ B cell as a % of total regulatory B cells; (i) decreased IgD+ CD27− CD19+ B cells as a % of total regulatory B cells; (j) decreased concentration of taurine; (k) increased concentration of aspartic acid; (l) increased concentration of phenylalanine; (m) increased concentration of alpha-ketoglutaric acid; (n) increased concentration of fumaric acid; (o) increased concentration of a lysophosphatidylcholine (LYSOC) selected from one or more of: LYSOC14:0; LYSOC16:1; and LYSOC20:3; and (p) decreased concentration of phosphatidylcholine (PC) PC40:6AA; as compared to a control non-cGVHD or to a non-cGVHD standard at an equivalent time period post HSCT.

The patient may be assigned a diagnosis of cGVHD where the biological sample has: (a) decreased CD56^{Bright} CD355⁺ CD56; CD56^{Bright} Perforin^{Negative} CD56; or CD56^{Bright} Granzyme B^{Negative} CD56 as a % of total lymphocytes; (b) increased CD56^{lo} P⁺ CD56; CD56^{lo} CD69⁺ CD56; or CD56^{lo} G⁺ CD56 as a % of total lymphocytes; (c) increased CD3+ T cells as a % of total lymphocytes; (d) increased PD1++ CD45RA+CD4+ T cells as a % of total lymphocytes; (e) decreased CD31− CD45RA+ CD4+ as a % of CD4+ cells; (f increased PD1+ CD45RA+CD8+ T cells as a % of total lymphocytes; (g) decreased CD19+ lymphocyte cells as a % of lymphocyte cells; (h) decreased transitional B cell selected from one or more of: a CD38− CD10− CD19+ B cell; a CD38^{dim} CD10^{lo} CD19+ B cell; and a CD38^{int} CD10^{int} CD19+ B cell as a % of total regulatory B cells; (i) decreased concentration of taurine; (j) increased concentration of aspartic acid; (k) increased concentration of phenylalanine; (l) increased concentration of alpha-ketoglutaric acid; (m) increased concentration of fumaric acid; (n) increased concentration of a lysophosphatidylcholine (LYSOC) selected from one or more of: LYSOC16:1; and LYSOC20:3; and (o) decreased concentration of phosphatidylcholine (PC) PC40:6AA; as compared to a control non-cGVHD or to a non-cGVHD standard at an equivalent time period post HSCT.

The biological sample may be obtained from a pediatric patient. The biological sample may be obtained from an adult patient. The biological sample may be obtained from the patient at one or more of: +100 days from HSCT (±14 days); 6 months from HSCT (±1 month); and 12 months from HSCT (±1 month).

The biological sample may be selected from the group consisting of: whole blood; plasma; and tissue. The detecting of the expression level of a biomarker may be via one or more of the following assay methods: flow cytometry; microarray analysis; immunoassay; immunohistochemistry; enzymatic assay; and mass spectrometry. The immune assay may be selected from: enzyme-linked immunoassay (ELISA); and meso scale.

The patient's cGVHD risk assignment may be as follows: (a) a high risk for a weighted sum of >2.1618; (b) a low risk for a weighted sum of <−0.0890; and (c) an intermediate risk for a weighted sum between −0.0890 and 2.1618.

The high risk patient may be preferentially administered an IST selected from one or more of: cyclosporine; voclosporin; tacrolimus; pimecrolimus; sirolimus; mycophenolate mofetil; mycophenolate sodium; ibrutinib; ruxolitinib; belomosudil; thalidomide; azathioprine; pentostatin; daclizumab; infliximab; and rituximab; or a biosimilar thereof. The high risk patient may be further administered a steroid treatment.

The steroid treatment may be selected from one or more of: prednisone; methylprednisolone; dexamethasone; beclomethasone; and budesonide. The low risk patient may be preferentially chosen for reduced duration of IST prophylaxis or early discontinuation of IST.

### BRIEF DESCRIPTION OF THE DRAWINGS

FIG. 1 shows mixed effect regression results of individual cellular and plasma biomarkers. Biomarker values at the onset of cGVHD were compared against blood samples from patients without cGVHD across all time points combined with blood samples from cGVHD patients before the onset of cGVHD. Dashed horizontal lines correspond to the Bonferroni corrected p-value threshold. Dashed vertical lines indicate the log 10 of the lower and upper limits of the effect ratio criterion. A dot (as opposed to a “x”) indicates the ROC AUC is above 0.6. (A) Onset of cGVHD of all severities (mild, moderate, severe) according to the NIH consensus criteria. Various populations of Naïve Helper T cells (Th), Naïve Regulatory T cells, Regulatory NK cells and cytolytic NK cells were decreased in cGVHD, whereas various cytokines and chemokines including CXCL9, CXCL10, CXCL11, ST2, ICAM-1, and enzymatic activity in sCD13 (aminopeptidase N) were increased at the onset of cGVHD (data not shown). (B) Onset of cGVHD restricted to cases meeting NIH consensus criteria for moderate to severe cGVHD (mild cases removed). Similar patterns of cellular and plasma biomarkers are present in moderate-severe cGVHD, with the exception that an additional population of NK_{REGS} is decreased (CD56^{BRIGHT}CD3^{NEGATIVE}Granzyme B^{NEGATIVE}) and decreased cytolytic NK cells are no longer significant (data not shown).

FIG. 2 shows fixed effect regression of individual cellular and plasma biomarkers. cGVHD patients were divided into groups based on days post-HSCT of cGVHD onset and compared to time-matched controls. (A) Early-onset of cGVHD before 4-months post-HSCT were compared to control biomarkers at day +100. (B) Mid-onset of cGVHD between 4-8 months post-HSCT were compared to control biomarkers at 6-months post-HSCT. (C) Later-onset of cGVHD between 8-12 months post-HSCT were compared to control biomarkers at 12-months post-HSCT. Dashed horizontal lines correspond to the nominal p-value threshold of 0.05. Dashed vertical lines indicate the log 10 of the lower and upper limits of the effect ratio criterion. Circled and labelled dots represent cell and plasma biomarkers that met our criteria in both the mixed effect and across all three time points in the fixed effect models.

FIG. 3 shows cGVHD diagnostic classifier. (A) Selection frequency of cellular and plasma markers based on all samples plotted. Markers with selection frequency>0.99 indicated in red and selection frequency>0.90 indicated in orange. (B) The classifier achieved an average ROC AUC of 0.89 over the 1,000 random sample splits.

FIG. 4 shows the classification performance with various combinations of clinical, cellular, plasma cytokines/chemokines, and metabolomics. (A) AUC of different data type combinations. Average AUC over 1000 random train-test samples splits plotted. Error bars correspond to standard deviation. (B) Positive predictive value of different combinations of data types. (C) Negative predictive value of different combinations of data types.

FIG. 5 shows mixed effect regression of individual cellular and plasma biomarkers in subsets of pediatric cGVHD. (A) Pulmonary cGVHD (n=12). (B) De Novo cGVHD (n=7). (C) Progressive cGVHD (including all cases of overlap syndrome) (n=18).

FIG. 6 shows day 100 cGVHD risk assignment algorithm. Selection frequency of cellular and plasma markers based on all samples plotted. Markers with selection frequency>0.70 indicated in red and selection frequency>0.60 indicated in orange. The classifier achieved an average ROC AUC of 0.78 over the 1,000 random sample splits.

FIG. 7 shows the percentage of subjects assigned by the risk assignment algorithm as having low risk, intermediate risk and high risk of developing cGVHD after day 114 post HSCT.

FIG. 8 shows a schematic diagram comparing the day 100 cGVHD risk assignment algorithm with the treatment assignment protocol.

FIG. 9 shows a schematic diagram of how the cGVHD diagnostic classifier and risk assignment algorithm were constructed.

FIG. 10 shows baseline characteristics. ALL, Acute Lymphoblastic Leukemia; AML, Acute Myelogenous Leukemia; HLA, Human Leukocyte Antigen; MDS, Myelodysplastic Syndrome.

FIG. 11 shows mixed effect modeling of cellular and plasma chronic GVHD diagnostic biomarkers (present at the onset of chronic GVHD) compared to control individuals without chronic GVHD. Chronic GVHD patients included all chronic GVHD severities (mild, moderate, severe) according to the NIH consensus criteria. Biomarkers highlighted in blue also met the same criteria at all three time points in the fixed effect model. Biomarkers highlighted in yellow met the same criteria in one or two time points (but not all three time points) in the fixed effect model. AUC, Area Under Curve; NK_{REG}, Regulatory NK cell; RTE, Recent Thymic Emigrant (CD4⁺CD45RA⁺CD31⁺); Th, Helper T cell (CD4⁺); T_{REG}, Regulatory T cell (CD4⁺CD127^{Low}CD25⁺).

FIG. 12 shows mixed effect modeling of cellular and plasma chronic GVHD diagnostic biomarkers (present at the onset of chronic GVHD) in moderate-severe chronic NIH consensus criteria GVHD (n=34) compared to control individuals without chronic GVHD. Patients with mild chronic GVHD according to the NIH consensus criteria were removed from analysis. AUC, Area Under Curve; NK_{REG}, Regulatory NK cell; RTE, Recent Thymic Emigrants (CD4⁺CD45RA⁺CD31⁺); Th, Helper T cell (CD4⁺); T_{REG}, Regulatory T cell (CD4⁺CD127^{Low}CD25⁺).

FIG. 13 shows fixed effect modeling of cellular and plasma chronic GVHD diagnostic biomarkers (present at the onset of chronic GVHD) compared to all control individuals without chronic GVHD. Chronic GVHD patients included all chronic GVHD severities (mild, moderate, severe), according to the NIH consensus criteria. Markers highlighted in blue met these three criteria at all three time points of chronic GVHD onset (early-, mid-, and late-onset). Markers highlighted in yellow met the three criteria at one or two (but not all three) time points for chronic GVHD onset. AUC, Area Under Curve; NK_{REG}, Regulatory NK cell; RTE, Recent Thymic Emigrants (CD4⁺CD45RA⁺CD31⁺); Th, Helper T cell (CD4⁺); T_{REG}, Regulatory T cell (CD4⁺CD127^{Low}CD25⁺).

FIG. 14 shows exploratory mixed effect modeling of cellular and plasma chronic GVHD diagnostic biomarkers (present at the onset of chronic GVHD) by type of chronic GVHD compared to all control individuals without chronic GVHD. All three criteria to be a biologically relevant potential diagnostic biomarker had to be met, including: (1) an effect ratio≥1.3 or ≤0.75, meaning the mean percentage in the chronic GVHD group had to either 30% greater or 30% lower (1.0/1.3=≤0.75) compared to the mean value of the control group; (2) the area under the curve (AUC) on the receiver operator curve had to be ≥0.60; and (3) p<0.05. None of the markers met the p-value criteria for Bonferroni correction. AUC, Area Under Curve; NK_{REG}, Regulatory NK cell; RTE, Recent Thymic Emigrants (CD4⁺CD45RA⁺CD31⁺); Th, Helper T cell (CD4⁺); T_{REG}, Regulatory T cell (CD4⁺CD127^{Low}CD25⁺).

### DETAILED DESCRIPTION OF THE INVENTION

The following detailed description will be better understood when read in conjunction with the appended figures. For the purpose of illustrating the invention, the figures demonstrate embodiments of the present invention. However, the invention is not limited to the precise arrangements, examples, and instrumentalities shown.

### Definitions

Any terms not directly defined herein shall be understood to have the meanings commonly associated with them as understood within the art of the invention.

As used herein the term “biomarker” or “biological marker” is any measurable indicator of a biological state. Biomarkers as used herein may be either molecular biomarkers (i.e. chemokines, cytokines, metabolites, enzymes) or cellular biomarkers. These biomarkers may have a clinical role in narrowing or guiding treatment decisions, assigning the risk, making a diagnosis or confirming a clinical suspicion. Biomarkers as described herein, may be useful in assigning the risk of developing cGVHD before onset or confirming a clinical suspicion to make a cGVHD diagnosis. In particular, predictive biomarkers may provide a 100 day risk assignment as either high, intermediate or low risk of developing cGVHD, where the biomarkers have either a selection frequency of ≥60% or ≥70%. Alternatively, the diagnostic biomarkers may provide a diagnostic classification of whether a patient has cGVHD as opposed to L-aGVHD or no GVHD, where the biomarkers have either a selection frequency of ≥90% or ≥99%.

As used herein the term “diagnostic biomarker panel” as used herein is a collection of 2 or more “biomarkers” or “biological markers” used to narrow or guide treatment decisions, assignment of risk, to make a diagnosis or confirm a clinical suspicion. Biomarker panels as described herein, may be useful to assign a risk of developing cGVHD before onset or to confirm a clinical suspicion to make a cGVHD diagnosis. In particular, predictive biomarker panels may provide a 100 day risk assignment as either high, intermediate or low risk of developing cGVHD. The “diagnostic biomarker panels” described herein may be chosen to improve on selectivity and/or specificity over individual “biomarkers” or “biological markers” or known biomarker panels. As used herein “sensitivity” refers to the biomarker panel's ability to correctly detect patients who do have cGVHD whereas “specificity” relates to biomarker panel's ability to correctly reject healthy individuals without cGVHD or L-aGVHD.

As used herein the term “diagnostic biomarker panel” is meant to include:
a NK_{REG} Non-Cytolytic cell selected from one or more of: CD56^{Bright} CD355⁺ CD56 (CD56^{hi} CD355⁺ CD56); CD56^{Bright} Perforin^{Negative} CD56 (CD56^{hi} P^{lo} CD56); and CD56^{Bright} Granzyme B^{Negative} CD56 (CD56^{hi} G^{lo} CD56);
a Cytolytic NK cell that is CD56^{lo} Granzyme B^{hi} CD56 (CD56^{lo} G⁺ CD56);
a Naïve Helper T cell selected from one or more of: CD4+ CD45RA+ CD3 (CD3+CD45RA+ T cell); CCR7+ CD45RA+ CD4+ T cell (CCR7⁺ CD45RA⁺ CD4+); PD1-CD45RA+ CD4+ T cell (PD1⁻ CD45RA⁺ CD4+); CD27+ CD45RA+ CD4+ T cell (CD27⁺ CD45RA⁺ CD4+); and CD31+ CD45RA+ CD4+ T cell (CD31⁺ CD45RA⁺ CD4+);
a Memory T Helper Cell that is CCR7− CD45RA− CD4+ T cell (CCR7⁻ CD45RA⁻ CD4+);
a Naïve Cytotoxic T Cell selected from one or more of: CCR7+ CD45RA+ CD8+ T cell (CCR7⁺ CD45RA⁺ CD8); and CD27+ CD45RA+ CD8+ T cell (CD27⁺ CD45RA⁺ CD8);
a Naïve Regulatory T Cell selected from one or more of: PD1− CD45RA+ Treg; and CD31+ CD45RA+ Treg;
a C-X-C motif chemokine ligand selected from one or more of: CXCL9; CXCL10; and CXCL11;
an intercellular adhesion molecule-1 (ICAM-1);
a T-cell immunoglobulin and mucin domain-3 (TIM-3);
a suppression of tumorigenicity 2 (ST2); and
a matrix metalloproteinase-3 (MMP-3).

Alternatively, the term “diagnostic biomarker panel” may include: a NK_{REG} Non-Cytolytic cell selected from one or more of: CD56^{Bright} CD355⁺ CD56 (CD56^{hi} CD355⁺ CD56); CD56^{Bright} Perforin^{Negative} CD56 (CD56^{hi} P^{lo} CD56); and CD56^{Bright} Granzyme B^{Negative} CD56 (CD56^{hi} G^{lo} CD56); a Cytolytic NK cell that is CD56^{lo} Granzyme B^{hi} CD56 (CD56^{lo} G⁺ CD56); a Naïve Helper T cell selected from one or more of: CCR7+ CD45RA+ CD4+ T cell (CCR7⁺ CD45RA⁺ CD4+); PD1− CD45RA+ CD4+ T cell (PD1⁻ CD45RA⁺ CD4+); CD27+ CD45RA+ CD4+ T cell (CD27⁺ CD45RA⁺ CD4+); and CD31+ CD45RA+ CD4+ T cell (CD31⁺ CD45RA⁺ CD4+); a Memory T Helper Cell that is CCR7− CD45RA− CD4+ T cell (CCR7⁻ CD45RA⁻ CD4⁺); a Naïve Cytotoxic T Cell selected from one or more of: CCR7+ CD45RA+ CD8+ T cell (CCR7⁺ CD45RA⁺ CD8); and CD27+ CD45RA+ CD8+ T cell (CD27⁺ CD45RA⁺ CD8); a Naïve Regulatory T Cell selected from one or more of: PD1-CD45RA+ Treg; and CD31+ CD45RA+ Treg; a C-X-C motif chemokine ligand selected from one or more of: CXCL9; CXCL10; and CXCL11; an intercellular adhesion molecule-1 (ICAM-1); a T-cell immunoglobulin and mucin domain-3 (TIM-3); a suppression of tumorigenicity 2 (ST2); and a matrix metalloproteinase-3 (MMP-3).

Alternatively, the term “diagnostic biomarker panel” may include: a NK_{REG} Non-Cytolytic cell selected from one or more of: CD56^{Bright} Perforin^{Negative} CD56 (CD56^{hi} P^{lo} CD56); and CD56^{Bright} Granzyme B^{Negative} CD56 (CD56^{hi} G^{lo} CD56); a Naïve Helper T cell selected from one or more of: CCR7+ CD45RA+ CD4+ T cell (CCR7⁺ CD45RA⁺ CD4+); PD1− CD45RA+ CD4+ T cell (PD1⁻ CD45RA⁺ CD4+); CD27+ CD45RA+ CD4+ T cell (CD27⁺ CD45RA⁺ CD4+); and CD31+ CD45RA+ CD4+ T cell (CD31⁺ CD45RA⁺ CD4+); a Naïve Cytotoxic T Cell that is CCR7+ CD45RA+ CD8+ T cell (CCR7⁺ CD45RA⁺ CD8); a Naïve Regulatory T Cell selected from one or more of: PD1− CD45RA+ Treg; and CD31+ CD45RA+ Treg; a C-X-C motif chemokine ligand selected from one or more of: CXCL9; CXCL10; and CXCL11; an intercellular adhesion molecule-1 (ICAM-1); a T-cell immunoglobulin and mucin domain-3 (TIM-3); and a suppression of tumorigenicity 2 (ST2).

As used herein the term “risk assignment biomarker panel” is meant to include:
a CD19+lymphocyte;
a transitional B cell selected from one or more of: a CD38− CD10− CD19+ B cell (CD38⁻ CD10⁻ CD19); a CD38^{dim} CD10^{lo} CD19+ B cell (CD38^{dim} CD10^{lo} CD19); and a CD38^{int} CD10^{int} CD19+ B cell (CD38^{int} CD10^{int} CD19);
an NK_{REG} Non-Cytolytic cell selected from one or more of: a CD56^{hi} CD355^{hi} CD56 (CD56^{hi} CD355⁺ NK); a CD56^{hi} Perforin^{Negative} CD56 (CD56^{hi} P^{lo} NK); and a CD56^{hi} Granzyme B^{Negative} CD56 (CD56^{hi} G^{lo} NK);
a Cytolytic NK cell selected from one or more of: a CD56^{dim} P^{hi} CD56 (CD56^{lo} P⁺ NK);
a CD56^{dim} CD69+CD56+ (CD56^{lo} CD69⁺ NK); and a CD56^{lo} G^{hi} CD56 (CD56^{lo} G⁺ NK);
a CD3+ lymphocyte (CD3+ T cell);
a PD1++CD45RA+ CD4+ Follicular T cell (PD1⁺⁺CD45RA⁺ CD4);
a PD1+ CD45RA+ CD8+ T cell (PD1⁺ CD45RA⁺ CD8);
a CD31− CD45RA+ CD4+ T cell (CD31⁻ CD45RA⁺ CD4);
a mature B cell that is IgD+ CD27− CD19+ B cell (IgD⁺ CD27⁻ CD19);
a lysophosphatidylcholine (LYSOC) selected from one or more of: LYSOC14:0; LYSOC16:1; and LYSOC20:3;
a Taurine;
an Aspartic acid;
a Phenylalanine;
an alpha-Ketoglutaric acid;
a Fumaric acid; and
a phosphatidylcholine (PC) PC40:6AA.

Alternatively, the term “risk assignment biomarker panel” may include: a CD19+ lymphocyte; a transitional B cell selected from one or more of: a CD38− CD10− CD19+ B cell (CD38⁻ CD10⁻ CD19); a CD38^{dim} CD10^{lo} CD19+ B cell (CD38^{dim} CD10^{lo} CD19); and a CD38^{int} CD10^{int} CD19+ B cell (CD38^{int} CD10^{int} CD19); an NK_{REG} Non-Cytolytic cell selected from one or more of: a CD56^{hi} CD355^{hi} CD56 (CD56^{hi} CD355⁺ NK); a CD56^{hi} Perforin^{Negative} CD56 (CD56^{hi} P^{lo} NK); and a CD56^{hi} Granzyme B^{Negative} CD56 (CD56^{hi} G^{lo} NK); a Cytolytic NK cell selected from one or more of: a CD56^{dim} P^{hi} CD56 (CD56^{lo} P⁺ NK); a CD56^{dim} CD69+CD56+ (CD56^{lo} CD69⁺ NK); and a CD56^{lo} G^{hi} CD56 (CD56^{lo} G⁺ NK); a CD3+ lymphocyte (CD3+ T cell); a PD1++CD45RA+ CD4+ Follicular T cell (PD1⁺⁺CD45RA⁺ CD4); a PD1+ CD45RA+ CD8+ T cell (PD1⁺ CD45RA⁺ CD8); a CD31− CD45RA+ CD4+ T cell (CD31⁻ CD45RA⁺ CD4); a lysophosphatidylcholine (LYSOC) selected from one or more of: LYSOC16:1; and LYSOC20:3; a Taurine; an Aspartic acid; a Phenylalanine; an alpha-Ketoglutaric acid; a Fumaric acid; and a phosphatidylcholine (PC) PC40:6AA.

As used herein the term “hi” is equivalent to “bright” in the context of biomarker descriptors. Similarly, the term “lo” is equivalent to “dim”” in the context of biomarker descriptors.

The risk assignment algorithm may allow for one of two things:
a. Identification of a high risk group with a 60% probability that later cGVHD will develop. This allows for an early preemptive application of immune suppression therapy (IST), such as ruxolitinib or rituximab, or prolonged used of the current prophylactic agents, including mycophenalate mofetil, tacrolimus, cyclosporine, and ruxolitinib, which is expected to decrease the severity or onset of cGVHD. Furthermore, such predictions could be useful in clinical trials to focus evaluation of a new therapy in the highest risk group.
b. The low risk group (6% probability of developing later cGVHD) may have a shortening duration of immune suppression therapy (IST) prophylaxis or early discontinuation of IST, which would minimize the potential side effects of IST.

As used herein “immune suppression therapy” or immunosuppressive therapy” (IST) refers to treatments that attenuate a patient's immune system modulate (i.e. the allo-immune response), control GVHD symptoms, and prevent organ damage or further organ damage. cGVHD symptoms may include one or more of the following: skin complaints (such as dryness, rash, itching, peeling, darkening, hard texture and feeling tight); dry eyes; dry mouth (with or without mouth ulcers); diarrhea; loss of appetite; stomach cramps; vomiting; weight loss; muscles pain; joint pain; increased infection; and difficulty breathing.

A clinical practitioner may use calcineurin inhibitors or other ISTs more readily for late aGVHD and use less calcineurin inhibitors for cGVHD. Late aGVHD is generally considered a less severe form of GVHD, than cGVHD.

As used herein, the term “calcineurin inhibitor” refers to compounds or compositions that are able to inhibit the action of calcineurin. Calcineurin is an enzyme that activates T-cells of the immune system (T-lymphocytes) that play a key role in cell-mediated immunity. Since calcineurin inhibitors suppress the immune system they are generally referred to as immunosuppressants. Commonly used calcineurin inhibitors include: cyclosporine, voclosporin, tacrolimus, and pimecrolimus.

For cGVHD, a number of agents are used almost always in combination with prednisone. These include, the immunosuppressant sirolimus, which is a rapamycin kinase (mTOR) inhibitor, that inhibits activation of T cells and B cells by reducing their sensitivity to interleukin-2 (IL-2) and the immunosuppressants are mycophenolate mofetil and mycophenolate sodium, which are inosine monophosphate dehydrogenase inhibitors.

Previous approaches have used either thalidomide or azathioprine, alone or in combination with prednisone. Thalidomide has immunosuppressive activity and acts through modulating the release of inflammatory mediators like tumor necrosis factor-alpha (TNF-α) and other cytokines. Azathioprine is an immunosuppressive agent, which functions through modulation of rac1 to induce T cell apoptosis.

Similarly, pentostatin may be used alone or in combination with other ISTs. Pentostatin is a potent inhibitor of adenosine deaminase (ADA). ADA activity is concentrated in lymphoid system cells. Given the high ADA activity in T-cells pentostatin acts as a T-cell inhibitor.

Also, the combination daclizumab and infliximab have been used to treat cGVHD and steroid refractory aGVHD.

In 2017, the FDA approved the first drug for cGVHD, ibrutinib which is a small molecule that acts as an irreversible potent inhibitor of Burton's tyrosine kinase (BTK) and BTK inhibition has a role in the B-cell receptor signaling. Ibrutinib may be used for refractory cGVHD. Since then ruxolitinib, a JAK1/2 inhibitor and belomosudil, a ROCK2 inhibitor have been approved for the treatment of cGVHD by the FDA. A CSFR1 inhibitor is showing promise in recent trials still underway.

One of the current and significant limitations in cGVHD is the need to use different immune suppressive agents in combination with steroids (i.e. prednisone, methylprednisolone, dexamethasone, beclomethasone and budesonide), which also have immune suppressive activity. The long term complications of steroids include irreversible osteonecrosis and growth inhibition in children. Accordingly, a major goal for future cGVHD therapy is to develop strategies that either eliminate steroids entirely or that minimize the long term side effects.

Many of these immune suppressive drugs, including steroids, have long term and sometimes irreversible toxicity. It is clear that early identification and intervention at the time of cGVHD and or preemption that might decrease its severity, is the optimal approach. While there have been excellent preventive strategies developed in the last 10 years that have decreased the frequency of cGVHD, including alemtuzumab, anti-thymocyte globulin post transplant cyclophosphamide, and ex vivo manipulations to deplete either TCRa/b T cells in combination with B cells or to deplete CD45RA+ naïve T cell, cGVHD still occurs. Biomarkers in the algorithms used can have two applications to minimize the impact of cGVHD. The risk assignment algorithm applied at day 100 post HSCT can identify patients at a high or low risk after their HSCT, but before the onset of cGVHD, which then can allow a preemptive addition of an immune suppressive strategy to further decrease the risk of developing cGVHD. The other is a cGVHD diagnostic classifier that allows for a definitive and early initiation of therapy for the optimal initiation of immune suppression therapy before cGVHD has the ability to progress and develop irreversible changes.

The risk assignment biomarkers measured at day 100 are capable of identifying low, moderate and risk patients with in 1 week of measurement. Thus allowing for a preemptive intervention in the high risk patients with an additional immune suppression treatments to decrease the risk of developing cGvHD. This improves on a prophylactic approach, where all patients receive additional immune suppression, which results in over treatment of low risk patients.

An “effective amount” of a pharmaceutical immunosuppressive treatments, as described herein includes a therapeutically effective amount or a prophylactically effective amount. A “therapeutically effective amount” refers to an amount effective, at dosages and for periods of time necessary, to achieve the desired therapeutic result, such as reduced cGVHD, and/or increased patient survival. A therapeutically effective amount of a compound may vary according to factors such as the disease state, age, sex, weight, risk assignment, and/or diagnostic determination of the patient, and the ability of the compound to elicit a desired response in the s patient. Dosage regimens may be adjusted to provide the optimum therapeutic response. A therapeutically effective amount is also one in which any toxic or detrimental effects of the compound are outweighed by the therapeutically beneficial effects. A “prophylactically effective amount” refers to an amount effective, at dosages and for periods of time necessary, to achieve the desired prophylactic result. Typically, a prophylactic dose is used in subjects prior to or at an earlier stage of disease, so that a prophylactically effective amount may be less than a therapeutically effective amount.

As used herein, the term “hematopoietic stem cell transplant” (HSCT) is sometimes also more generally referred to as “hematopoietic cell transplant” (HCT) involves the transplantation (i.e. intravenous infusion) of hematopoietic stem cells (i.e. derived from bone marrow, peripheral blood, or umbilical cord blood) in order to reestablish blood cell production in patients whose bone marrow or immune system is damaged or defective. The hematopoietic stem cells may be autologous (the patient's own stem cells), allogeneic (the stem cells from a donor) or syngeneic (from an identical twin). HSCT is used to treat certain cancers of the blood or bone marrow including multiple myeloma or leukemia. Furthermore, prior to HSCT chemotherapy or radiation may be given as a conditioning regimen, to help eradicate the patient's disease prior to the infusion of HSCs and to suppress the immune patient's response. However, HSCT and pre-HSCT conditioning can result in infection and GVHD.

### Materials and Methods

### Patients and Study Design

Between August 2013-February 2017, allogeneic HSCT patients 18 years of age were enrolled before the start of conditioning and prospectively followed for 1-year post-HSCT for the development of acute GVHD (aGvHD, onset before day +100), L-aGvHD (onset after day +100), and cGVHD (onset at any time after HSCT). All 27 transplant centers (6 Canadian, 20 United States, 1 Austrian) had research ethics board approval for the study and informed consent/assent was obtained for all participants in accordance with the Declaration of Helsinki. Any transplant indication (except second transplants) and all graft sources, conditioning regimen, and GVHD prophylaxis regimens were included. Detailed clinical assessments were performed, and case report forms completed at day +100 (±14 days), 6-months (±1 month), and 12-months (±1 month), with emphasis on GVHD status. Of the 302 patients enrolled on ABLE/PBMTC 1202, 234 were evaluable for this analysis, including 44 with cGVHD and 190 controls. Baseline characteristics of patients are not shown.

### GVHD Definitions and Groups

aGVHD was defined as an erythematous rash, nausea, vomiting, diarrhea, and hyperbilirubinemia occurring before day +100 and was staged and graded according to the modified Glucksberg criteria.¹⁹ Updated MAGIC™ aGVHD grading criteria were not published when the ABLE study opened.²⁰ L-aGVHD was defined as the same manifestations after day +100 in the absence of cGVHD. cGVHD was defined according to the 2005 NIH-CC, since the 2014 NIH-CC were not yet published when the study opened.¹¹ Centers completed a detailed cGVHD case report form in near real-time after a cGVHD diagnosis, documenting the clinical manifestations of cGVHD and severity according to NIH-CC, with a follow-up form at 1-year post-HSCT. Each submitted cGVHD case was reviewed by the site PI, centrally by the study PI, and when necessary, by a central study adjudication committee of cGVHD experts. Six of the 44 (13.6%) cGVHD patients did not meet formal NIH-CC for cGVHD diagnosis; however, after central review by the adjudication committee, were assessed as having manifestations that would be considered reasonably due to cGVHD and were included as cGVHD cases. Details have previously been published by our group.¹⁴ At the end of the study, patients were divided into: (1) a cGVHD group, consisting of patients developing cGVHD in the first year post-HSCT; or (2) a control group, consisting of patients with either no evidence of any GVHD or aGvHD and/or L-aGvHD only in the first year. Patients with overlap syndrome (concurrent aGvHD and cGVHD features at the onset of cGVHD) were included in the cGVHD group. The study groups were chosen as such to emphasize the clinical scenario of attempting to differentiate clinical and/or laboratory manifestations as being due to either cGVHD or a non-cGVHD cause (whether aGVHD, L-aGVHD, or a non-alloreactive etiology).

### Blood Samples

Peripheral blood samples were collected at day +100 (±14 days), 6-months (±1 month), and 12-months (±1 month) post-HSCT in all patients, and at the onset of a new cGVHD diagnosis before escalation of immunosuppression specifically to treat cGVHD (diagnostic cGVHD onset blood sample). Details of study blood sampling have previously been published.¹⁷

### Immunophenotyping and Cytokine Measurement

Six flow cytometric antibody panels consisting of combinations of cell surface markers were used to delineate 76 subpopulations of T cells, regulatory T cells (TREGS, CD4+CD127LowCD25+), B cells (including T1 transitional B cells: CD19+ CD38^{HIGH}CD10^{HIGH}; T2 transitional B cells: CD19+ CD38^{INT} CD10^{INT}; T3 transitional B cells: CD19+ CD38^{DIM}CD10^{LOW}; mature naïve B cells: CD19+ CD38-veCD10-ve IgD+ CD27−; unswitched memory B cells: CD19+IgD+CD27+; and class-switched memory B cells CD19+ IgD-ve CD27+), NK cells (including regulatory NK cells, NK_{REGS}), and myeloid cells. Cell subsets were measured as a percentage of their parent cell type.

Seven plasma cytokines and chemokines were analyzed by ELISA (measured as concentrations), including soluble BAFF, soluble CD25 (sIL2Rα), ICAM-1, CXCL10 (IP10), TIM-3, ST2, and MMP-3. CXCL9 and CXCL11 were measured using an electrochemiluminescence dual-plex plate (Meso Scale Diagnostics™, Gaithersburg, MD). Soluble CD13 (sCD13, aminopeptidase N) was measured in the plasma by enzymatic assay. Reg3α could not be performed due to hemolysis in some samples, affecting the accuracy of the assay. Details of all assays have been previously published.¹⁷ Completeness of analyzable control samples was 91.6% (day +100), 96.3% (6-months), and 87.7% (12-months) for cellular analysis by flow cytometry; and 95.8% (day +100), 90.4% (6-months) and 82.8% (12-months) for plasma cytokines and chemokines.

### Statistical Analysis

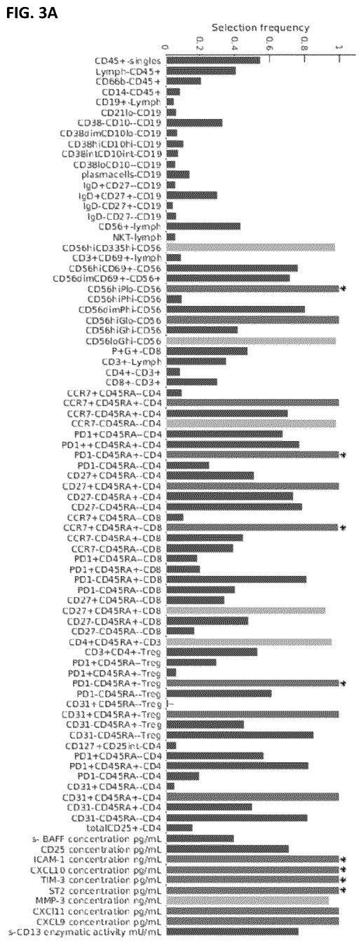

In addition to analyzing each marker in a univariate manner, we developed a machine learning-based classifier that combines multiple cellular and plasma markers along with clinical factors for diagnosing whether a patient has cGVHD. The approach is summarized in FIGS. 3A, 3B and FIG. 9. We first randomly selected 10 cGVHD samples and 10 control samples as the test set for classifier evaluation. If a control sample was from one of the selected cGVHD subjects (i.e., a measurement made prior to cGvHD onset) or another sample had already been drawn from the same control subject, we randomly drew another control sample, since in real clinical settings, we would immediately diagnose a subject when marker measurements become available. All remaining samples, except those acquired at later time points from subjects in the test set, were used for classifier training. To account for outlier marker values, we performed winsorization by first estimating the median and median absolute deviation (MAD) of each marker based on the training samples. We then clipped all marker values at 3 standard deviations away from the training median with standard deviation estimated as 1.483-MAD.⁴¹ To deal with missing marker values in the training set, we applied k-nearest neighbors (k=15) to impute the missing values. To reduce the number of markers, we performed feature selection using a bootstrapping approach. Specifically, we extracted 1,000 bootstrap samples from the training set. For each bootstrap sample, we applied a Student's t-test to compare the marker values of cGVHD samples against controls for each marker. Markers with p<0.05 for >99% of the bootstrap samples (i.e., selection frequency>0.99) were selected for classifier training. We trained a support vector machine (SVM) classifier with the selected markers and all clinical factors used in the regression analysis. The SVM found an optimal weighting of the selected markers and clinical factors that best separates cGVHD samples from control samples. To account for class imbalance (i.e., the training set had many more control samples than cGVHD samples), we set the penalty weight for misclassifying cGvHD (controls) to the number of training samples over the number of cGvHD (control) samples. Setting a higher penalty for misclassifying cGVHD reduces the bias towards classifying samples as controls. To deal with missing marker values in the test set, we applied k-nearest neighbors (k=15) using only values of the selected markers from training samples to impute the missing values in each test sample. Imputing each test sample separately without using other test samples better emulates real clinical settings. To evaluate the classifier, we applied it to the test set and computed its positive predictive value (PPV), negative predictive value (NPV), and receiver operator characteristic (ROC) area under the curve. PPV is the proportion of test samples classified as cGvHD that are truly cGvHD, and NPV is the proportion of test samples classified as controls that are truly controls. The described procedures were repeated 1,000 times to assess variability in classification performance across different sample splits. To generate a single classifier for future validation, we applied the same bootstrapping approach to all samples to select markers, which were then used along with the clinical factors to train a SVM. A cGVHD risk assignment algorithm was similarly built and evaluated except case samples correspond to measurements from cGVHD subjects prior to onset at 100 days after HSCT and control samples correspond to measurements from non-cGVHD subjects at 100 days after HSCT.

For the analysis to meet clinical practice needs (differentiate true cGVHD from aGVHD, L-aGVHD and other causes that mimic GVHD manifestations), we compared biomarkers from blood samples taken at the onset of cGVHD (experimental group) against biomarkers from patients in the control group. Regularly scheduled blood samples from cGVHD patients drawn before cGVHD onset (i.e., at day +100 and 6-months, if cGVHD had not yet developed by this time point) were treated as control samples, provided the blood samples were collected>14 days before the diagnosis of cGVHD.

We first evaluated individual cellular and plasma biomarkers at the onset of cGVHD diagnosis. For the main analysis, we applied a mixed effect linear regression model to contrast marker values of cGVHD onset samples against control samples at all measured time points. Subject-specific intercepts and number of days post-HSCT to blood collection were included as random effects to account for within-subject correlations. Confounding factors considered in this analysis included: recipient age; malignant vs. non-malignant disease; graft type (peripheral blood stem cells, bone marrow, or umbilical cord); sibling vs. unrelated donors; donor and recipient sex, HLA and ABO match/mismatch; myeloablative vs. reduced-intensity conditioning; and the use of serotherapy (anti-thymocyte globulin and alemtuzumab) and total body irradiation. An individual biomarker was considered relevant if three criteria were met (all had to be present): (1) an effect ratio≥1.3 or ≤0.75; (2) receiver operating characteristic (ROC) area under the curve (AUC)≥0.60; (3) the p-value had to be less than the Bonferroni corrected threshold (p<5.814×10-4). The effect ratio was estimated as the mean marker value of cGVHD samples over that of the control group. ROC AUC was computed by estimating the true positive rate (proportion of cGVHD correctly classified) against the false positive rate (proportion of controls falsely classified as cGVHD) for different marker thresholds.

A secondary analysis of individual biomarkers using fixed effect linear regression models was performed to explore the effect of differing days of cGVHD onset post-HSCT. For this analysis, cGVHD subjects were divided into early- (<4 months), mid-(4-8 months), and late-onset (>8 months) and compared against time-matched control samples at day 100, 6-months, and 12-months, respectively. Identical criteria for defining relevant markers were applied in the fixed effect analysis as in the mixed effect analysis, except p<0.05 was used to provide a more encompassing view of the plasma and cellular biomarker patterns (considering the lower statistical power with reduced sample size in the fixed effect analysis).

Given the pleomorphic nature of cGVHD (and hence a single marker might not be adequate to capture the variability across all patients for cGVHD diagnosis), we developed a clinically applicable machine learning-based classifier that combines multiple cellular and plasma markers, along with clinical factors, for determining whether a patient has cGVHD (or not). Clinical factors included: malignant vs. non-malignant disease; recipient age; graft type (PBSC, bone marrow, or umbilical cord); donor and recipient sex, HLA and ABO match/mismatch; donor source (sibling or unrelated); myeloablative vs. reduced-intensity conditioning; the use of serotherapy and total body irradiation; and days post-HSCT. The classifier training and evaluation procedures, where samples were first divided into training and test sets. Bootstrap univariate marker selection was then performed by applying t-test to the training samples of each marker and estimating the percentage of bootstraps over which a given marker has p<0.05, referred to as selection frequency, f. The set of markers, S, with selection frequency>0.99 were used for classifier training. Labels of test samples, Ip, were then predicted using the trained classifier weights, w, and compared against the ground truth labels, lg, to evaluate the classifier's performance. This procedure was repeated 1,000 with random sample splits to assess variability in performance. First samples were divided into a test set (10 cGVHD and 10 control samples, randomly selected) and a training set (remaining samples) and performed marker selection on the training set using a bootstrapping approach. A marker was selected if it reached nominal significance for 99% of 1000 bootstrap samples (i.e., selection frequency>0.99). We then trained a support vector machine (SVM), which finds a linear weighting of the selected markers and clinical factors that best separates cGVHD from control samples of the training set. Lastly, we applied the trained SVM to left out test samples for classification evaluation. We repeated the procedure 1000 times to assess variability in performance across different sample splits. We also tested the addition of metabolomic markers from our recent study into the classifier.¹⁸ All analyses were performed using MATLAB™ (MathWorks™, Natwick, Mass, USA).

### EXAMPLES

### Example 1: Cellular and Plasma Diagnostic Biomarkers of Chronic GVHD

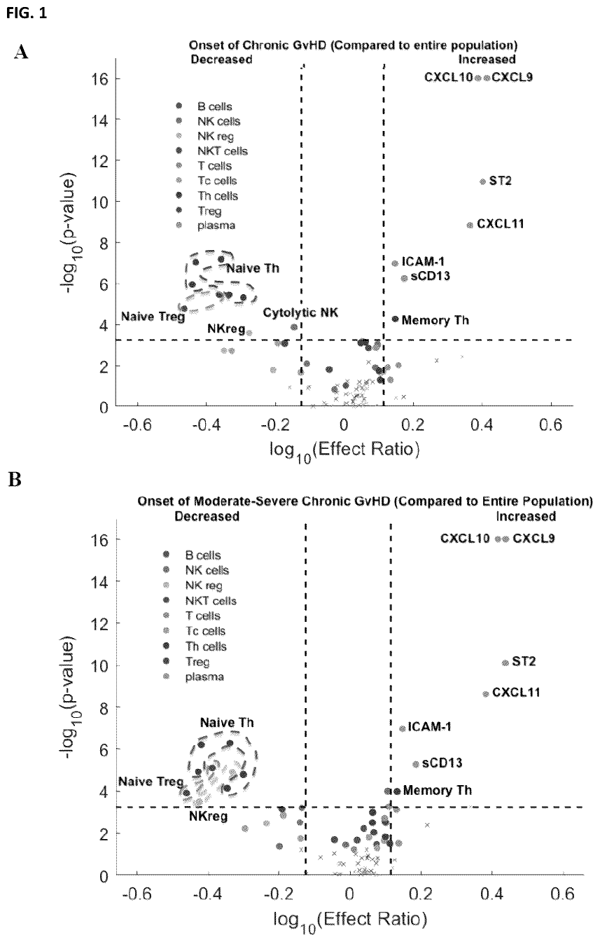

Contrasting the onset samples of pediatric cGVHD subjects against the control group using mixed effect analysis, cGVHD patients exhibited decreased proportions of CD56+NK cells (as a % of total lymphocytes), increased proportions of non-cytolytic regulatory NK cells (NK_{REGS}) (CD56^{Bright} CD3^{Negative} Perforin^{Negative}), four (4) populations of naïve helper T cells (CD4+CD45RA+CCR7+, CD4+CD45RA+PD1-ve, CD4+CD45RA+CD27+, CD4+CD45RA+CD31+, all as % of CD4+CD45RA+) and alternatively CD4+CD45RA+ as a % of total CD4+ cells; and two populations of naïve regulatory T cells (TREGS) (CD45RA+PD1-ve TREGS and CD45RA+CD31+ TREGS as % of total TREGS); along with increased proportions of effector memory Th cells (CD4+CD45RA-veCCR7-ve as % of CD4+). Elevated concentrations of CXCL9, CXCL10, CXCL11, ST2, and ICAM-1 in cGVHD patients relative to controls. Also, greater enzyme activity of sCD13 in cGVHD patients relative to controls was present (FIG. 1A and TABLE 2).

### Example 2: Evaluation of Biomarkers in NIH Moderate-Severe Pediatric Chronic GVHD

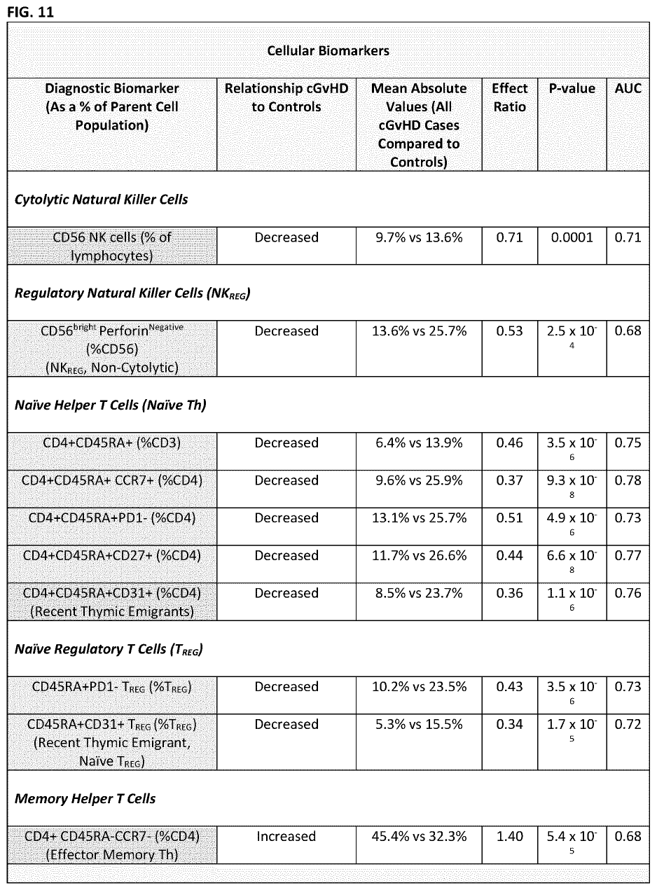

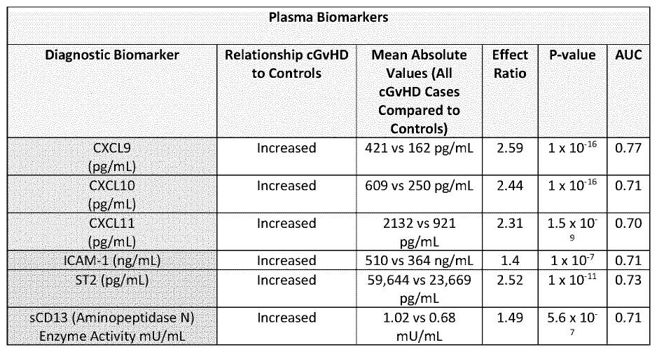

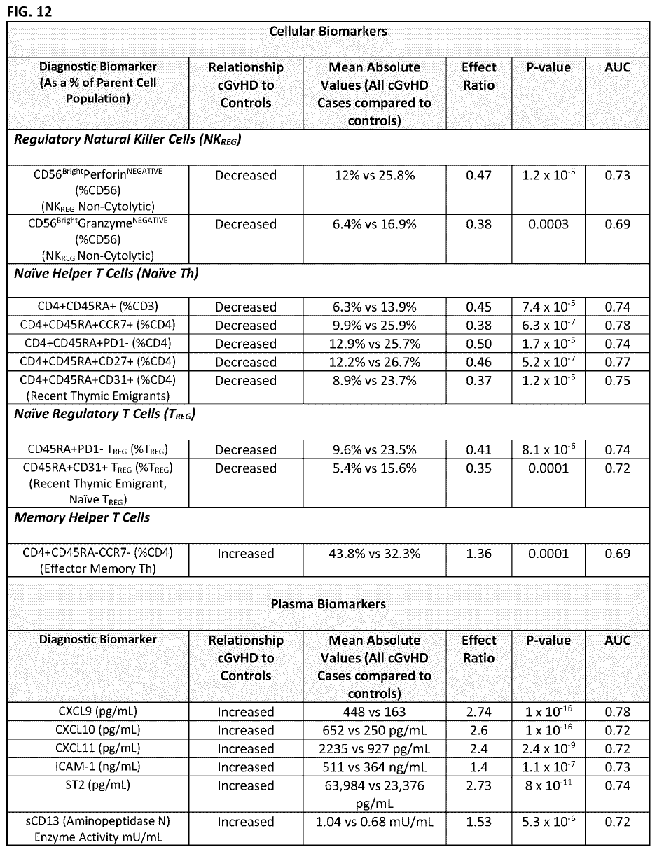

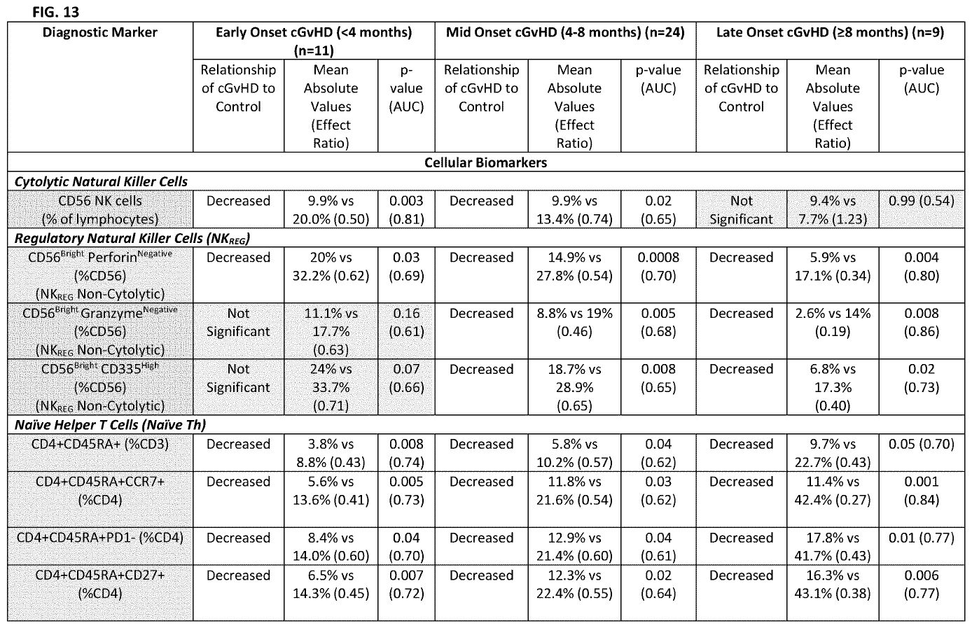

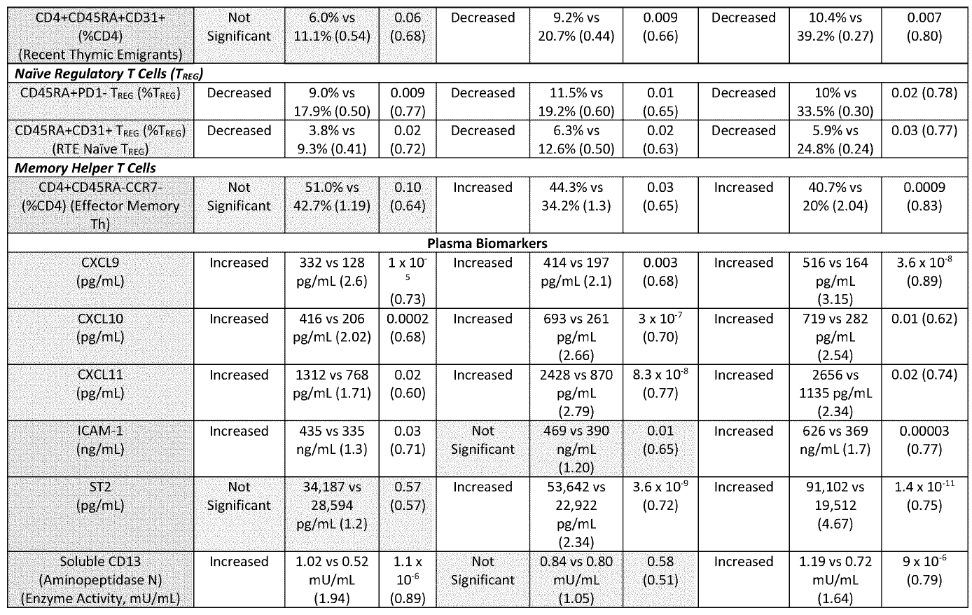

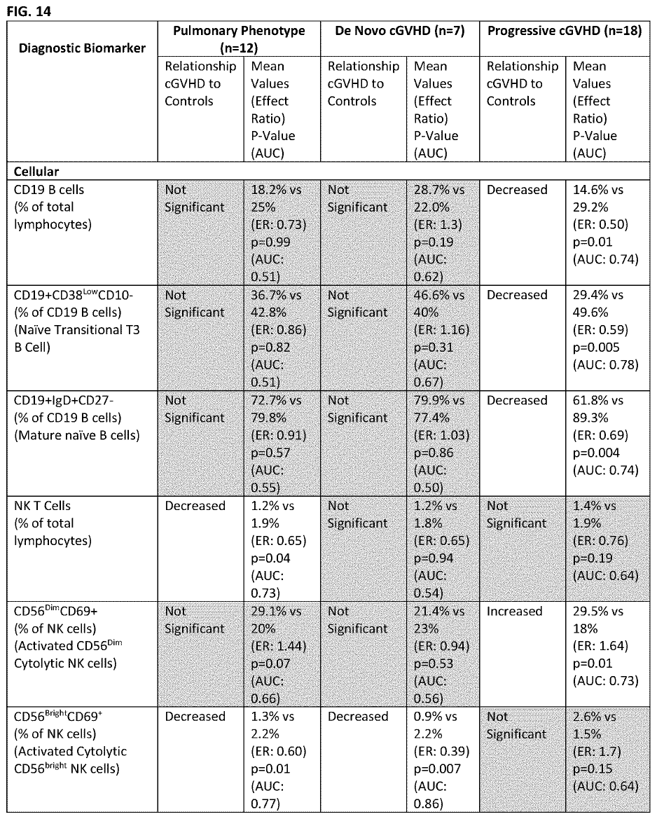

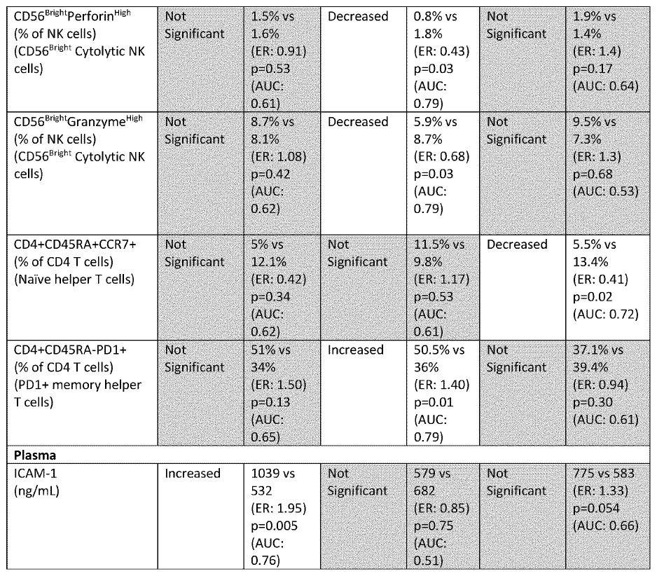

Given the clinical importance of moderate to severe NIH-CC cGVHD, we repeated the mixed effect analysis by including only those patients developing moderate to severe cGVHD in the first year post-HSCT. Similar patterns of cellular populations were found, except an additional population of NK_{REGS} (CD56^{Bright} CD3^{Negative} Granzyme B^{Negative}) were decreased in cGVHD, and cytolytic NK cells no longer met criteria. When the analysis included only NIH-CC moderate-severe cGVHD, CXCL9, CXCL10, CXCL11, ST2, and ICAM-1 and sCD13 remained elevated (FIG. 11B).

### Example 3: Evaluation of Time Dependence of Chronic GVHD Biomarker Expression

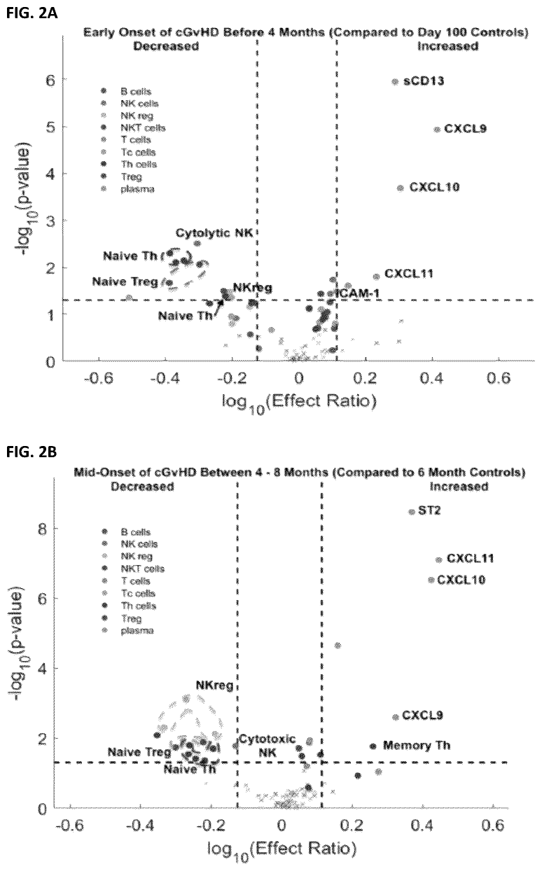

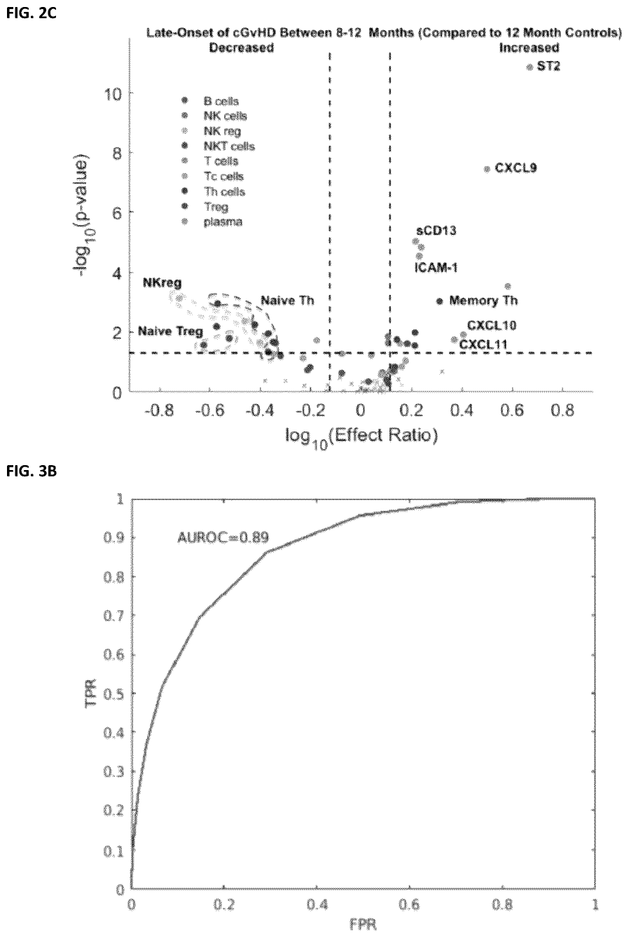

To evaluate whether biomarker changes were dependent upon the timing of onset of cGVHD after HSCT, we divided the cGVHD patients into three groups (early-, mid-, and late-onset) and performed a fixed effect analysis for each time point. Similar patterns of cellular biomarkers were found, although not all biomarkers retained their significance across all three time points. CXCL9, CXCL10, and CXCL11 maintained their significance across all three time points of cGVHD onset, but ICAM-1 and sCD13 were only elevated at the early- and late time points, and ST2 was elevated at the mid- and late time points (FIG. 2).

### Example 4: Combinations of Cellular, Plasma and Clinical Factors in Developing a Chronic GVHD Diagnostic Classifier

Nine cellular markers, including populations of NK_{REGS}, naïve Th cells, naïve TREGS, and naïve CD8+ cytotoxic T cells (all as percentages of their parent cell types); and six plasma cytokine and chemokine markers, including CXCL9, CXCL10, CXCL11, ICAM-1, TIM-3, and ST2 attained selection frequency>0.99 (FIG. 3A and TABLE 1). Combining these cellular and plasma markers with 11 clinical factors (see statistical analysis) into a cGVHD diagnostic classifier achieved an average AUC (over 1000 random test sets) of 0.89 (±0.07) (FIG. 31B), average positive predictive value (PPV) of 82% (±11%) and average negative predictive value (NPV) of 80% (±11%). Since some of the cellular and plasma cytokine and chemokine markers are interrelated and may therefore not all be required, we tested keeping only representative markers and removing the interrelated markers from the classifier: CXCL10 (representing CXCL9, CXCL10, and CXCL11); CD4+ CD45RA+ PD1− (as a marker of naïve helper CD4+ T cells); and CD56^{Bright} CD3^{Negative} Perforin^{Negative} (as a marker of NK_{REGS}). The AUC remained the same (0.89±0.07), suggesting the classifier could be reduced to six cellular markers (instead of nine) and four plasma cytokines and chemokines (instead of six). We also tested adding metabolomic markers from our recent study into the classifier pipeline,¹⁸ but the average AUC did not improve (AUC 0.88).

TABLE 1
Cellular and plasma chronic GVHD markers with a selection
frequency ≥0.99 in the diagnostic chronic GVHD classifier.

| | Immune Phenotype | Selection |
|---|---|---|
| Cell Type or Plasma Protein | or Plasma Protein | Frequency |
| NK_{REG} Non-Cytolytic | CD56^{Bright} Perforin^{Negative} | 0.999 |
| NK_{REG} Non-Cytolytic | CD56^{Bright} Granzyme B^{Negative} | 0.999 |
| Naïve Helper T Cells | CD4− CD45RA− PD1− | 1 |
| Naïve Helper T Cells | CD4− CD45RA+ CCR7+ | 1 |
| Naïve Helper T Cells | CD4+ CD45RA+ CD27− | 1 |
| Naïve Helper T Cells (RTE) | CD4+ CD45RA+ CD31− | 1 |
| Naïve Cytotoxic T Cells | CD8+ CD45RA+ CCR7+ | 0.994 |
| Naïve Regulatory T Cells | CD45RA+ PD1− T_{REG} | 1 |
| Naïve Regulatory T Cells (RTE) | CD45RA+ CD31+ T_{REG} | 1 |
| Plasma Protein | CXCL9 | 1 |
| Plasma Protein | CXCL10 (IP10) | 0.998 |
| Plasma Protein | CXCL11 | 1 |
| Plasma Protein | ICAM-1 | 1 |
| Plasma Protein | TIM-3 | 0.999 |
| Plasma Protein | ST2 | 1 |

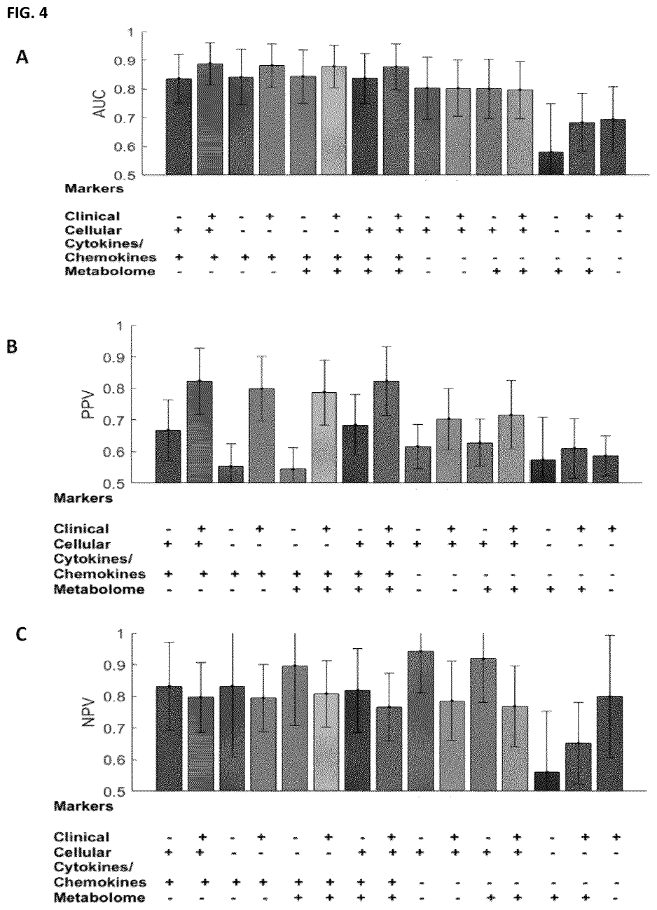

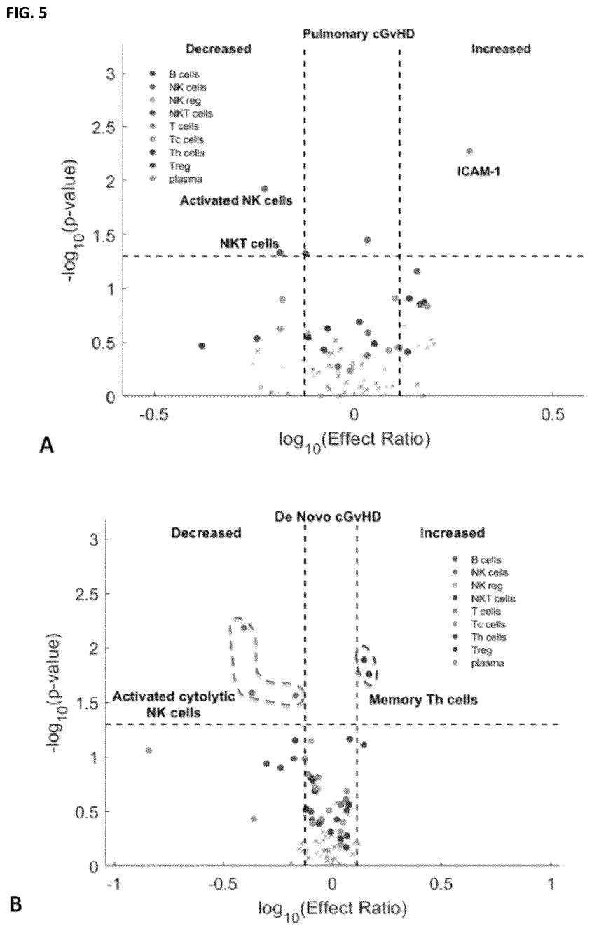

In clinical practice, a rapid turn-around time for classifier results would be most beneficial to patients and clinicians, given the necessity of making an accurate cGVHD diagnosis in near real-time. Since the plasma cytokines and chemokines in the classifier are assayed by ELISA and/or Meso Scale, if performed individually at the time of sample receipt (as opposed to being batched with multiple patient samples at a later point), complexity and expense with the classifier may increase, but we are investigating flow cytometry methods for testing. We therefore tested removing the plasma markers from the classifier and including only the 11 clinical factors and 9 cellular markers (which can be assayed quickly and inexpensively at the time of receipt by flow cytometry); the AUC, however, decreased to 0.80. Lastly, we compared various combinations of clinical factors, cellular markers, plasma cytokines/chemokines and metabolomics, along with their impact on the AUC, PPV, and NPV (FIG. 4). As evident from FIG. 4, the presented classifier comprising cellular and plasma cytokines/chemokines markers combined with clinical factors provides the best classification performance. We note that older patients who received peripheral blood stem cell grafts were more often misclassified (data not shown). The SVM classifier weight of each variable is not shown.

### Example 5: cGVHD Subset Analysis

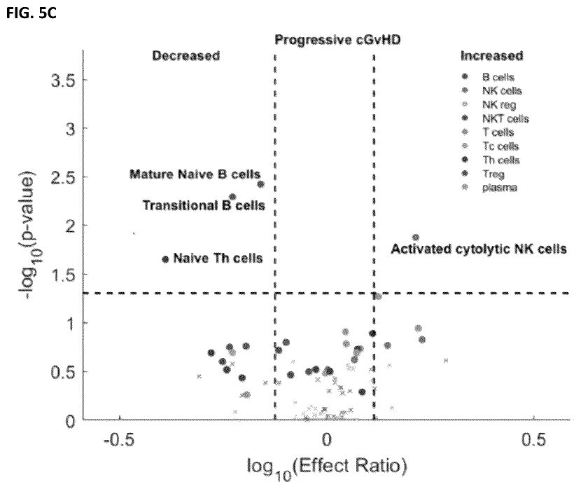

To better understand clinically relevant subtypes of cGVHD, a post-hoc exploratory analysis was performed by applying mixed effect modelling on individual biomarkers using diagnostic samples from cGVHD patients with a pulmonary cGVHD phenotype (n=12). Our group has previously published the challenges with diagnosing pediatric pulmonary cGVHD in this cohort.¹⁴ Patients were therefore included if they met NIH-CC, or if not, were still highly suspected of having pulmonary cGVHD. Decreases in NKT cells and activated cytolytic CD56^{bright} NK cells (CD56^{bright} CD69+) and increases in ICAM-1 were seen in pulmonary cGVHD (FIG. 5A). We also explored biomarkers seen in de novo cGVHD (no previous history of acute GVHD) (n=7) and progressive cGVHD (acute GVHD progressing into cGVHD, including overlap syndrome) (n=18). De novo cGVHD was characterized by decreased activated cytolytic NK cell populations (CD56^{bright}CD69+; CD56^{bright}Perforin^{High} and Granzyme B^{High}) and increases in memory helper T cells (FIG. 5B). Progressive cGVHD was characterized by decreased percentages of B cells, reductions in mature naïve B cells (CD19+ CD38-ve CD10-ve IgD+ CD27−), T3 transitional B cells (CD19+ CD38^{Dim} CD10^{Low}), and naïve helper T cells (CD4+ CD45RA+ CCR7+) with increases in activated CD56^{Dim} cytolytic NK cells (CD56^{Dim} CD69+) (FIGURE SC). Other cGVHD subsets, including by specific organ system, were not possible due to marked heterogeneity in clinical presentations and small patient numbers.

Using a prospective, multi-institutional study design, a well-characterized cohort of pediatric HSCT survivors with central adjudication of cGVHD status and strict biomarker criteria, the ABLE/PBTMC 1202 study demonstrates that cellular and plasma diagnostic cGVHD biomarkers are present at the onset of cGVHD in children and adolescents. Relevant biomarkers include decreased non-cytolytic regulatory NK cells (NK_{REGS}), naïve helper T cells, and naïve regulatory T cells (TREGS); increased effector memory Th cells; and increases in CXCL9, CXCL10, CXCL11, ICAM-1, ST2, and sCD13. These markers are present at the onset of cGVHD in patients who develop moderate to severe cGVHD according to the NIH-CC in the first year after HSCT. Some of these markers appear independent of the time post-HSCT when cGVHD is diagnosed (e.g., CXCL9, CXCL10, CXCL11), whereas others (e.g., ST2, sCD13, ICAM-1) may be time-dependent. We also found novel markers in pulmonary, de novo, and progressive cGVHD. Given the small numbers of patients in these subgroup analyses, however, an independent validation cohort would assist before making further conclusions.

Given the complex immunopathology and clinical heterogeneity of cGVHD, a single marker is unlikely sufficient for diagnosing all cGVHD cases. We therefore developed a machine learning-based cGVHD diagnostic classifier that incorporates multiple cellular, plasma, and clinical factors. The high AUC (0.89) suggests this classifier could aid clinicians in differentiating cGVHD at its initial onset from aGVHD, L-aGVHD, and other non-cGVHD manifestations. Using the classifier alone, however, could result in ~10% of subjects being misclassified on average. This emphasizes the necessity of clinicians still performing comprehensive cGVHD clinical assessments and using clinical judgement, both at the time of suspected cGVHD diagnosis and thereafter, while considering non-GVHD causes of various symptoms, signs, and investigations. The classifier is therefore complimentary to clinical evaluation, helping to achieve a more accurate cGVHD diagnosis. Interestingly, although the clinical factors used in classifier are mostly risk factors for cGVHD (e.g., use of PBSC vs other graft sources, HLA-match vs mismatch) as opposed to diagnostic or distinctive cGVHD signs, they improved classification performance. These clinical factors are easily obtainable data points in routine practice. Also, despite our previous publication showing elevated α-ketoglutaric acid levels both before and at the onset of cGVHD,¹⁸ adding metabolomic markers to the classifier did not improve the AUC. Furthermore, removing the plasma markers and including only the clinical factors and cellular markers, reduced the AUC to 0.80, suggesting the plasma markers are important for cGVHD classification. Our diagnostic classifier requires further validation in a new pediatric cohort before clinical application. The ABLE 2.0/PTCTC GVH-1901 study (NCT04372524) is currently open and enrolling pediatric patients and will attempt to do this. A secondary objective of this study will be to test the feasibility of performing both the plasma and cellular cGVHD biomarker assays with a 10-day turn-around time from receipt of blood sample to return of results to the clinician, an important consideration in developing a real-world application.

Important for clinical application is that when cGVHD developed, most of the patients in our cohort were either receiving GVHD prophylaxis or had recently been or were being treated with systemic immune suppression for aGVHD/L-aGVHD. Since patients are often immunosuppressed when cGVHD develops, clinical application of the diagnostic classifier is independent of this fact. For proper clinical translation, however, blood samples must be drawn when NIH-CC cGVHD is initially suspected or diagnosed and before further escalation of immunosuppression therapy to treat cGVHD (as was done in our data analysis).

Many of the diagnostic cGVHD biomarkers in this study have been previously described, lending validity to our findings. CD56^{bright} NK cells are mostly non-cytolytic NK_{REGS}, expressing low levels of granzyme B and perforin and appearing to regulate innate and adaptive immunity.²¹⁻²³ Consistent with our finding of decreased NK_{REGS}, low percentages of CXCR3+CD56^{bright} NK_{REGS} and elevations in CXCL10 (a chemokine important for trafficking CXCR3+ effector cells, including CD56^{bright} NK_{REGS}) have been documented in adult cGVHD.²⁴

Lower proportions of CD56bright NK_{REGS} in peripheral blood donor grafts have also been associated with higher cGVHD rates.²⁵

Interferon-γ-inducible CXCR3-binding chemokines CXCL9, CXCL10, and CXCL11 recruit type 1 helper T cells (Th1) and cytotoxic T cells (Tc1) to sites of inflammation and have well-appreciated roles in cGVHD.^{9,26-28} Elevations in these chemokines are increasingly reported as reproducible early diagnostic biomarkers of cGVHD.^{24,26,29-32} Our results from the ABLE/PBMTC 1202 study are consistent. Adults later developing severe cGVHD are noted to have elevated CXCL9 levels by day +100 post-HSCT,^{30,31} suggesting importance in the early inflammatory stages of cGVHD. ST230 and sCD1333 have also been previously documented as elevated at the time of cGVHD diagnosis.

One observation from the ABLE/PBMTC 1202 study is that pediatric cGVHD may be associated with diminished thymopoeisis. Recent thymic emigrants (CD4+CD45RA+CD31+)^{34,35} and TREGS recently emigrated from the thymus (CD31+CD45RA+ T_{REG})^{36,37} were decreased in our cGVHD cohort, and both were selected in the diagnostic classifier. Numerous factors influence thymopoeisis after HSCT, including age, sex, genetic factors, GVHD, and intensity of the conditioning regimen.³⁴ Since most of our patients received myeloablative regimens and we controlled for conditioning intensity and the use of total body irradiation, we hypothesize that the impact of pre-existing acute GVHD on the thymus may be an explanation.^{38,39} In this same cohort of pediatric ABLE patients, grades 2-4 aGVHD and age≥12 years (where thymic rebound post-HSCT might be less robust) were the two most important risk factors for developing cGHVD.¹⁴ This suggests efforts to protect thymic function in children and adolescents after HSCT may be particularly important for cGVHD prevention.

The ABLE study has several strengths, including its prospective study design, near real-time adjudication of cGVHD clinical features according to the NIH-CC (ensuring proper classification of patient cohorts and avoiding recall bias), the inclusion of multiple centers with low to high transplant volumes (real world representation), and blood samples drawn in the early stages of a cGVHD diagnosis before immune suppression is escalated. This classifier, however, requires a new validation cohort before broad clinical application. The next-generation ABLE 2.0 study, occurring in collaboration with the Pediatric Transplant and Cellular Therapy Consortium, will serve as a validation cohort for the diagnostic classifier developed here, potentially bringing diagnostic biomarkers of pediatric cGVHD closer to clinical utility.

### Example 6: a cGVHD Risk Assignment Algorithm at Day 100 Before the Onset of cGvHD with Cellular and Plasma Risk Assignment Biomarkers

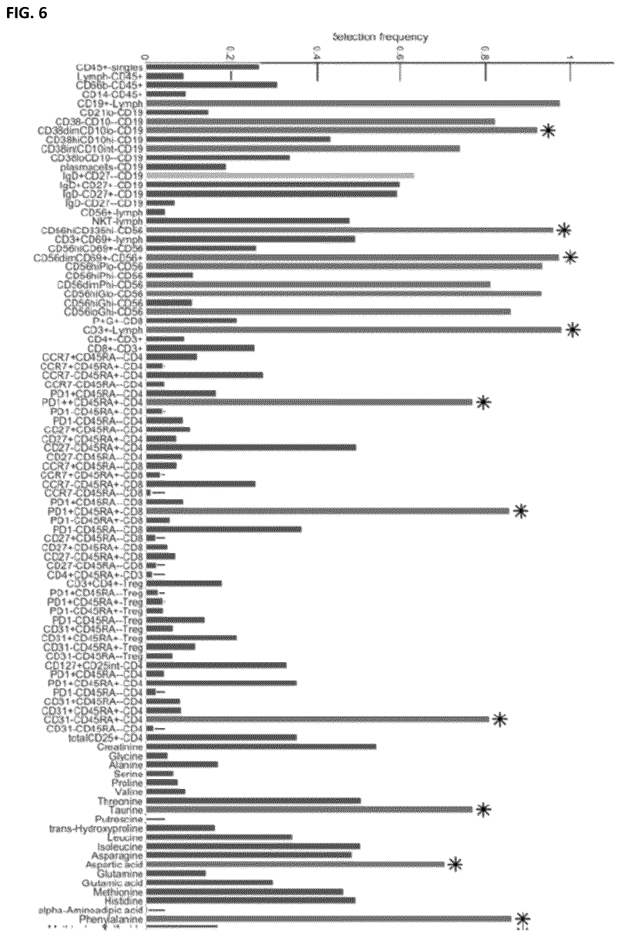

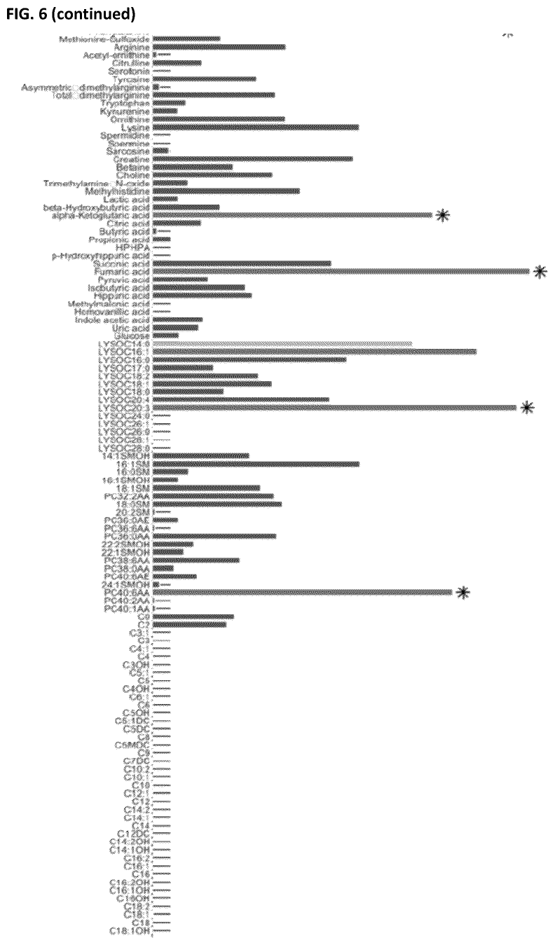

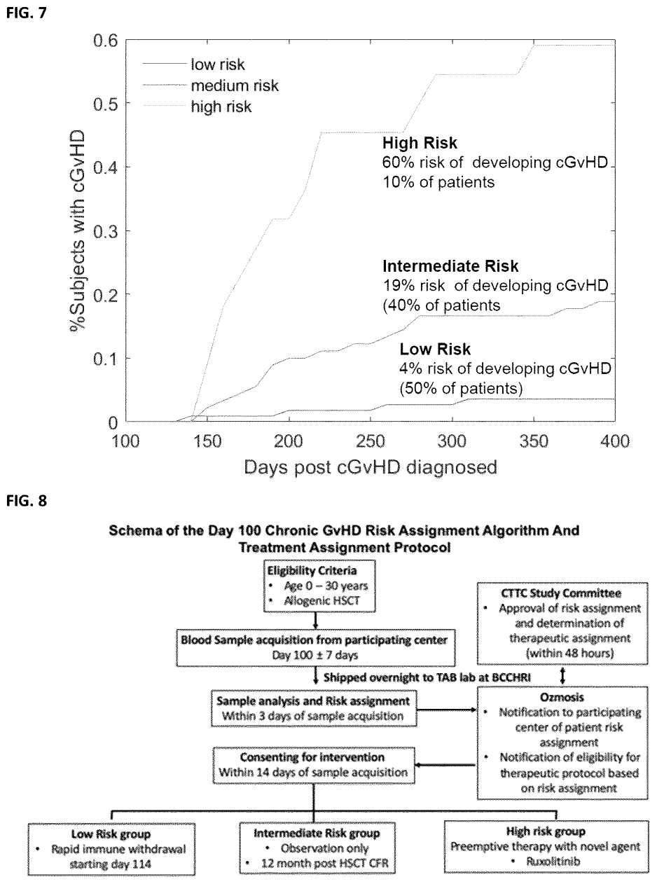

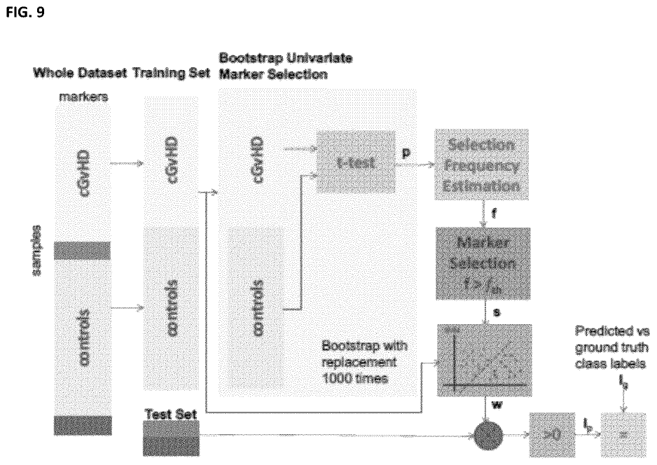

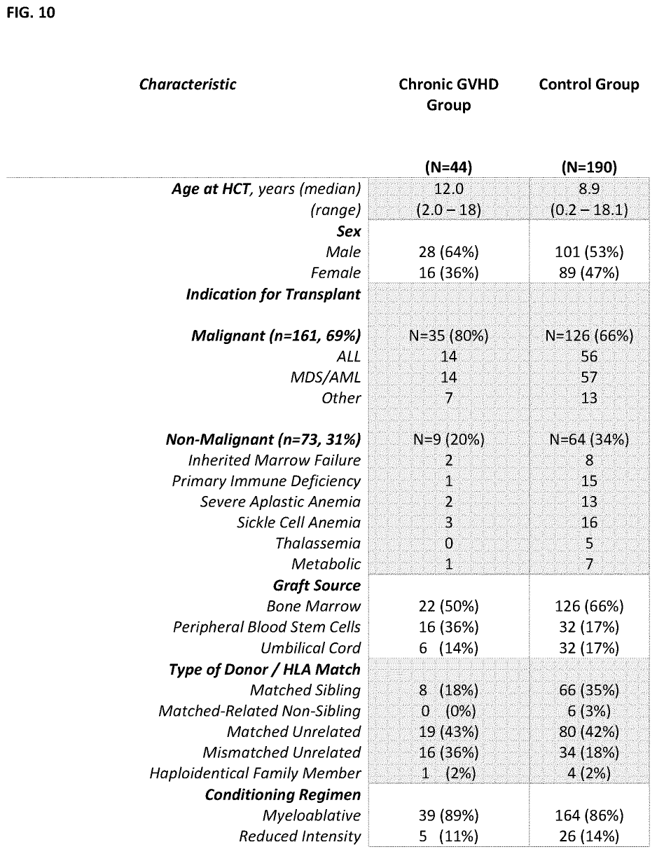

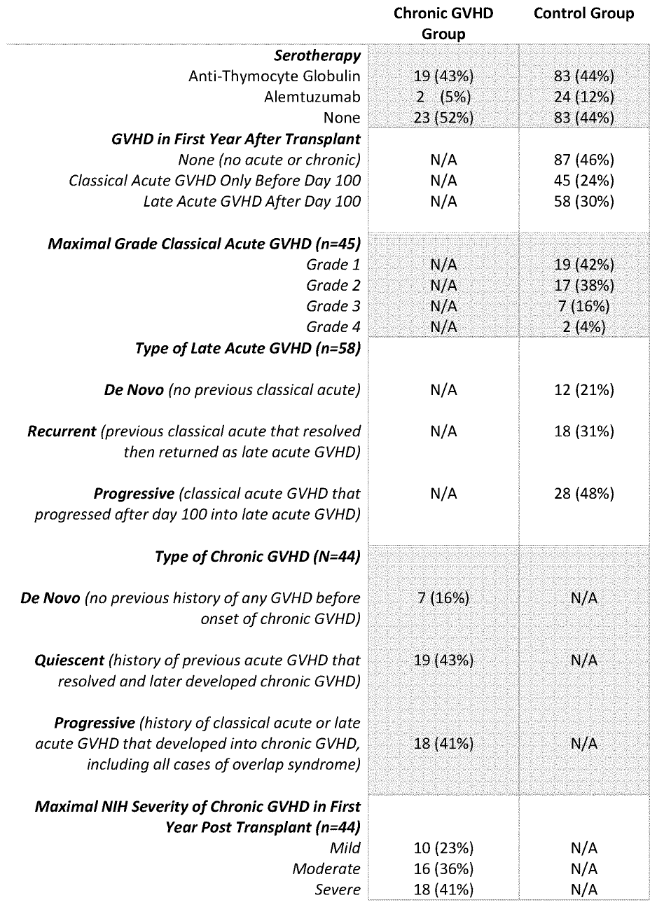

We developed a cGVHD risk assignment algorithm using the procedures summarized in FIG. 8. With a selection frequency threshold of 0.7, 14 cell populations and 8 metabolites were selected (FIG. 6). Training a SVM with these markers in combination with 11 clinical variables resulted in an average ROC AUC of 0.78, positive predictive value (PPV) of 0.74, and negative predictive value (NPV) of 0.68 over 1000 random sample splits. Applying this risk assignment algorithm under a leave-one-out cross-validation framework (FIG. 7) identified 10% of the patients as high-risk (59% of which developed cGvHD), 40% of patients as moderate-risk (19% of which developed cGvHD), and 50% of the patients as low-risk (4% of which developed cGvHD).

We attempted to develop a diagnostic algorithm using each marker in isolation (see TABLE 2), but at best, a ROC AUC of 0.78 was attained with CCR7+ Naïve Th cells or CXCL9 alone. We thus instead adopted a polyomic approach using machine learning to develop a diagnostic algorithm (FIG. 6). With a selection frequency threshold of 0.99, 9 cell populations and 6 cytokines were selected (FIG. 7). Training a SVM with these markers in combination with 11 clinical variables resulted in an average ROC AUC of 0.89, PPV of 0.82, and NPV of 0.77 over 1000 random sample splits.

Pediatric cGVHD patients exhibited decreased proportions of CD56+ NK cells (as a % of total lymphocytes), three increased populations of cytolytic regulatory NK cells (NK_{REGS}) (CD56^{lo}P+NK; CD56^{lo}CD69⁺ NK; and CD56^{lo}G⁺ NK), a decreased population of naïve helper T cells (CD31⁻CD45RA⁺CD4⁺T cell, all as % of CD4+CD45RA+); an increased population of naïve CD8+ T cells (PD1⁺CD45RA⁺CD8⁺ T cell as % of total TREGS); and a decreased population of naïve regulatory T cells (TREGS) (CD19+Lymphocytes as % of total naïve TREGS). Along with decreased concentrations of Taurine and PC40:6AA, with increased concentrations of aspartic acid, phenylalanine, alpha-KG, fumaric acid, LYSOC14:0, LYSOC16:1, and LYSOC20:3 in cGVHD patients relative to controls were also present (FIGS. 6-7 and TABLE 2).

TABLE 2
Summary of Risk Assignment and Diagnostic Markers
Table summarizing the markers Selection Frequency used in Chronic
GvHD Day 100 risk assignment and Diagnostic algorithms

| | Diagnostic Classifier - | | | Day 100 Risk Assignment | | |
|---|---|---|---|---|---|---|
| | Selection frequency | | | Algorithm - Selection frequency | | |
| | | | Non- | | | Non- |
| | | | redundant | | | redundant |
| Marker | ≥90% | ≥99% | markers | ≥60% | ≥70% | markers |

NKreg cells (group together)

| | | | | | | |
|---|---|---|---|---|---|---|
| CD56hiCD335+ NK | − | | | − | − | − |
| CD56hiPloNK cells | − | − | − | − | − | |
| CD56hiGloNK | − | − | | − | − | |

Classic/cytolytic NK cells (group together)

| | | | | |
|---|---|---|---|---|
| CD56loP+ NK cell | | + | + | |
| CD56loCD69+ NK cell | | + | + | + |
| CD56loG+ NK cell | + | + | + | |
| CD3+ T cell | | + | + | + |

Follicular T cells

| | | | |
|---|---|---|---|
| PD1++ CD45RA+ | + | + | + |
| CD4+ T cell | | | |

Naïve T helper cells (group together)

| | | | | | | |
|---|---|---|---|---|---|---|
| CD4+ CD45RA+ CD3 cell | − | | | | | |
| collective for category of | | | | | | |
| naïve T helper cells | | | | | | |
| OR CD3+ CD45RA+ T cell | | | | | | |
| OR CD45RA+ CD4+ T | | | | | | |
| CCR7+ CD45RA+ CD4+ T cell | − | − | | | | |
| PD1− CD45RA+ CD4+ T cell | − | − | − | | | |
| CD31+ CD45RA+ CD4+ T cell | − | − | | | | |
| CD27+ CD45RA+ CD4+ T cell | − | − | | | | |
| CD31− CD45RA+ CD4+ T cell | | | | − | − | − |

Memory T helper cells

| | |
|---|---|
| CCR7− CD45RA− CD4+ T cell | + |

Naïve CD8+ T cells (group together)

| | | | | | | |
|---|---|---|---|---|---|---|
| CCR7+ CD45RA+ CD8+ T cell | − | − | − | | | |
| CD27+ CD45RA+ CD8+ T cell | − | | | | | |
| PD1+ CD45RA+ CD8+ T cell | | | | + | + | + |

Naïve Treg cells (group together)

| | | | | | |
|---|---|---|---|---|---|
| PD1− CD45RA+ Treg | − | − | − | | |
| CD31+ CD45RA+ Treg | − | − | | | |
| CD19+_Lymph | | | | − | − |

Transitional B cells (group together)

| | | | |
|---|---|---|---|
| CD38− CD10− CD19+ B cell | − | − | |
| CD38dimCD10loCD19+ B cell | − | − | − |
| CD38intCD10intCD19+ B cell | − | − | |

Mature B cells

| | |
|---|---|
| IgD+ CD27− CD19+ B cell | − |

Chemokines (group together)

| | | | |
|---|---|---|---|
| CXCl11 | + | + | |
| CXCL10 | + | + | + |
| CXCL9 | + | + | |

Cytokines (none group together)

| | | | |
|---|---|---|---|
| ICAM1 | + | + | + |
| ST2 | + | + | + |
| TIM-3 | + | + | + |
| MMP3 | + | | |

Metabolites (group together)

| | | | | | | |
|---|---|---|---|---|---|---|
| Taurine | | | | − | − | − |
| Aspartic acid | | | | + | + | + |
| Phenylalanine | | | | + | + | + |
| Alpha-KG | | | | + | + | + |
| Fumaric acid | | | | + | + | + |
| LYSOC14:0 | | | | + | | |
| LYSOC16:1 | | | | + | + | |
| LYSOC20:3 | | | | + | + | + |
| PC40:6AA | | | | − | − | − |
| PPV | 0.82 | 0.82 | 0.81 | 0.75 | 0.74 | 0.73 |
| NPV | 0.78 | 0.80 | 0.81 | 0.66 | 0.68 | 0.68 |
| AUROC | 0.89 | 0.89 | 0.89 | 0.78 | 0.78 | 0.78 |
| AUCPR | 0.78 | 0.78 | 0.79 | 0.69 | 0.70 | 0.70 |

One of the outputs of the risk assignment algorithm is a score for each patient. The higher the score, the higher the chance of developing cGVHD. We assume 50% of the patients have low risk, 40% of the patients have medium risk, and the remaining 10% of patients have high risk based on this score. With this assumption, 4%, 19%, and 60% of patients in the low, medium, and high risk groups developed cGVHD, respectively. Note that the algorithm also provides a binary output of whether a patient is likely to develop cGVHD. The AUC of 0.78 is referring to this aspect of the algorithm.

The SVM performs a weighted sum of the marker values (after all the normalization steps) and clinical variables, and as a result the weights can be negative. A weighted sum of >2.1618 means that the patient is “high risk” (>2.1618=high risk); weighted sum of <−0.0890 means that the patient is “low risk” (<−0.0890=low risk); and anything in between means that the patient is at “intermediate risk”.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. The word “comprising” is used herein as an open-ended term, substantially equivalent to the phrase “including, but not limited to”, and the word “comprises” has a corresponding meaning. As used herein, the singular forms “a”, “an” and “the” include plural referents unless the context clearly dictates otherwise. Thus, for example, reference to “a thing” includes more than one such thing. Citation of references herein is not an admission that such references are prior art to an embodiment of the present invention. The invention includes all embodiments and variations substantially as hereinbefore described and with reference to the examples and drawings.

### REFERENCES

1. El-Jawahri A, Pidala J, Khera N, et al. Impact of Psychological Distress on Quality of Life, Functional Status, and Survival in Patients with Chronic Graft-versus-Host Disease. Biol Blood Marrow Transplant. 2018.
2. Pidala J, Kurland B, Chai X, et al. Patient-reported quality of life is associated with severity of chronic graft-versus-host disease as measured by NIH criteria: report on baseline data from the Chronic GVHD Consortium. Blood. 2011; 117(17):4651-4657.
3. Krupski C, Jagasia M. Quality of Life in the Chronic GVHD Consortium Cohort: Lessons Learned and the Long Road Ahead. Curr Hematol Malig Rep. 2015; 10(3):183-191.
4. Eapen M, Horowitz M M, Klein J P, et al. Higher mortality after allogeneic peripheral-blood transplantation compared with bone marrow in children and adolescents: the Histocompatibility and Alternate Stem Cell Source Working Committee of the International Bone Marrow Transplant Registry. J Clin Oncol. 2004; 22(24):4872-4880.
5. Armenian S H, Sun C L, Kawashima T, et al. Long-term health-related outcomes in survivors of childhood cancer treated with HSCT versus conventional therapy: a report from the Bone Marrow Transplant Survivor Study (BMTSS) and Childhood Cancer Survivor Study (CCSS). Blood. 2011; 118(5):1413-1420.
6. Bhatia S, Francisco L, Carter A, et al. Late mortality after allogeneic hematopoietic cell transplantation and functional status of long-term survivors: report from the Bone Marrow Transplant Survivor Study. Blood. 2007; 110(10):3784-3792.
7. Boyiadzis M, Arora M, Klein J P, et al. Impact of Chronic Graft-versus-Host Disease on Late Relapse and Survival on 7,489 Patients after Myeloablative Allogeneic Hematopoietic Cell Transplantation for Leukemia. Clin Cancer Res. 2015; 21(9):2020-2028.
8. Jacobsohn D A, Arora M, Klein J P, et al. Risk factors associated with increased nonrelapse mortality and with poor overall survival in children with chronic graft-versus-host disease. Blood. 2011; 118(16):4472-4479.
9. Cooke K R, Luznik L, Sarantopoulos S, et al. The Biology of Chronic Graft-versus-Host Disease: A Task Force Report from the National Institutes of Health Consensus Development Project on Criteria for Clinical Trials in Chronic Graft-versus-Host Disease. Biol Blood Marrow Transplant. 2017; 23(2):211-234.
10. Zeiser R, Blazar B R. Pathophysiology of Chronic Graft-versus-Host Disease and Therapeutic Targets. N Engl J Med. 2017; 377(26):2565-2579.
11. Filipovich A H, Weisdorf D, Pavletic S, et al. National Institutes of Health consensus development project on criteria for clinical trials in chronic graft-versus-host disease: I. Diagnosis and staging working group report. Biol Blood Marrow Transplant. 2005; 11(12):945-956.
12. Jagasia M H, Greinix H T, Arora M, et al. National Institutes of Health Consensus Development Project on Criteria for Clinical Trials in Chronic Graft-versus-Host Disease: I. The 2014 Diagnosis and Staging Working Group report. Biol Blood Marrow Transplant. 2015; 21(3):389-401 e381.
13. Cuvelier G D E, Schoettler M, Buxbaum N P, et al. Towards a Better Understanding of the Atypical Features of Chronic Graft-Versus-Host Disease: A Report from the 2020 National Institutes of Health Consensus Project Task Force. Transplant Cell Ther. 2022.
14. Cuvelier G D E, Nemecek E R, Wahlstrom J T, et al. Benefits and challenges with diagnosing chronic and late acute GVHD in children using the NIH consensus criteria. Blood. 2019; 134(3):304-316.
15. Paczesny S, Hakim F T, Pidala J, et al. National Institutes of Health Consensus Development Project on Criteria for Clinical Trials in Chronic Graft-versus-Host Disease: III. The 2014 Biomarker Working Group Report. Biol Blood Marrow Transplant. 2015; 21(5):780-792.
16. Adom D, Rowan C, Adeniyan T, Yang J, Paczesny S. Biomarkers for Allogeneic HSCT Outcomes. Front Immunol. 2020; 11:673.
17. Schultz K R, Kariminia A, Ng B, et al. Immune profile differences between chronic GVHD and late acute GVHD: results of the ABLE/PBMTC 1202 studies. Blood. 2020; 135(15):1287-1298.
18. Subburaj D, Ng B, Kariminia A, et al. Metabolomic Identification of Alpha-Ketoglutaric Acid Elevation in Pediatric Chronic Graft-versus-Host Disease. Blood. 2021.
19. Przepiorka D, Weisdorf D, Martin P, et al. 1994 Consensus Conference on Acute GVHD Grading. Bone Marrow Transplant. 1995; 15(6):825-828.
20. Harris A C, Young R, Devine S, et al. International, Multicenter Standardization of Acute Graft-versus-Host Disease Clinical Data Collection: A Report from the Mount Sinai Acute GVHD International Consortium. Biol Blood Marrow Transplant. 2016; 22(1):4-10.
21. Michel T, Poli A, Cuapio A, et al. Human CD56bright NK Cells: An Update. J Immunol. 2016; 196(7):2923-2931.
22. Cooper M A, Fehniger T A, Turner S C, et al. Human natural killer cells: a unique innate immunoregulatory role for the CD56(bright) subset. Blood. 2001; 97(10):3146-3151.
23. Fehniger T A, Cooper M A, Nuovo G J, et al. CD56bright natural killer cells are present in human lymph nodes and are activated by T cell-derived IL-2: a potential new link between adaptive and innate immunity. Blood. 2003; 101(8):3052-3057.
24. Kariminia A, Holtan S G, Ivison S, et al. Heterogeneity of chronic graft-versus-host disease biomarkers: association with CXCL10 and CXCR3+NK cells. Blood. 2016; 127(24):3082-3091.
25. Kariminia A, Ivison S, Ng B, et al. CD56(bright) natural killer regulatory cells in filgrastim primed donor blood or marrow products regulate chronic graft-versus-host disease: the Canadian Blood and Marrow Transplant Group randomized 0601 study results. Haematologica. 2017; 102(11):1936-1946.
26. Croudace J E, Inman C F, Abbotts B E, et al. Chemokine-mediated tissue recruitment of CXCR3+CD4+T cells plays a major role in the pathogenesis of chronic GVHD. Blood. 2012; 120(20):4246-4255.
27. Choi J, Ziga E D, Ritchey J, et al. IFNgammaR signaling mediates alloreactive T-cell trafficking and GVHD. Blood. 2012; 120(19):4093-4103.
28. Hakim F T, Memon S, Jin P, et al. Upregulation of IFN-Inducible and Damage-Response Pathways in Chronic Graft-versus-Host Disease. J Immunol. 2016; 197(9):3490-3503.
29. Kitko C L, Levine J E, Storer B E, et al. Plasma CXCL9 elevations correlate with chronic GVHD diagnosis. Blood. 2014; 123(5):786-793.
30. Yu J, Storer B E, Kushekhar K, et al. Biomarker Panel for Chronic Graft-Versus-Host Disease. J Clin Oncol. 2016; 34(22):2583-2590.
31. Giesen N, Schwarzbich M A, Dischinger K, et al. CXCL9 Predicts Severity at the Onset of Chronic Graft-versus-host Disease. Transplantation. 2020; 104(11):2354-2359.
32. Ahmed S S, Wang X N, Norden J, et al. Identification and validation of biomarkers associated with acute and chronic graft versus host disease. Bone Marrow Transplant. 2015; 50(12):1563-1571.
33. Fujii H, Cuvelier G, She K, et al. Biomarkers in newly diagnosed pediatric-extensive chronic graft-versus-host disease: a report from the Children's Oncology Group. Blood. 2008; 111(6):3276-3285.
34. Gaballa A, Clave E, Uhlin M, Toubert A, Arruda L C M. Evaluating Thymic Function After Human Hematopoietic Stem Cell Transplantation in the Personalized Medicine Era. Front Immunol. 2020; 11:1341.
35. Fink P J. The biology of recent thymic emigrants. Annu Rev Immunol. 2013; 31:31-50.
36. Matos T R, Hirakawa M, Alho A C, Neleman L, Graca L, Ritz J. Maturation and Phenotypic Heterogeneity of Human CD4+Regulatory T Cells From Birth to Adulthood and After Allogeneic Stem Cell Transplantation. Front Immunol. 2020; 11:570550.
37. Wagner M I, Mai C, Schmitt E, et al. The role of recent thymic emigrant-regulatory T-cell (RTE-Treg) differentiation during pregnancy. Immunol Cell Biol. 2015; 93(10):858-867.
38. Clave E, Busson M, Douay C, et al. Acute graft-versus-host disease transiently impairs thymic output in young patients after allogeneic hematopoietic stem cell transplantation. Blood. 2009; 113(25):6477-6484.
39. Krenger W, Hollander G A. The immunopathology of thymic GVHD. Semin Immunopathol. 2008; 30(4):439-456.
40. Cuvelier G D E, Li A, Drissler S, et al. Age Related Differences in the Biology of Chronic Graft-Versus-Host Disease After Hematopoietic Stem Cell Transplantation. Fronteiers in Immunology 2020 11:571884.
41. Gijbels, I. and Hubert M.: Robust and Nonparametric Statistical Methods, in Brown S D, Tauler R, and Walczak B (eds): Comprehensive Chemometrics. Chemical and Biochemical Data Analysis. Elseiver, 2009, volume 1, pp 189-211.

What is claimed is:

1. A chronic graft-versus-host disease (cGVHD) diagnostic biomarker panel, the panel comprising:
(a) a NK_{REG} Non-Cytolytic cell selected from one or more of: CD56^{Bright} Perforin^{Negative} CD56; and CD56^{Bright} Granzyme B^{Negative} CD56;
(b) a Naïve Helper T cell selected from one or more of: CCR7+ CD45RA+ CD4+; PD1− CD45RA+ CD4+; CD27+ CD45RA+ CD4+; and CD31+ CD45RA+ CD4+;
(c) a Naïve Cytotoxic T Cell that is CCR7+CD45RA+CD8+;
(d) a Naïve Regulatory T Cell selected from one or more of: PD1− CD45RA+ Treg; and CD31+ CD45RA+ Treg;
(e) a chemokine selected from one or more of: CXCL9; CXCL10; and CXCL11;
(f) an intercellular adhesion molecule-1 (ICAM-1);
(g) a T-cell immunoglobulin and mucin domain-3 (TIM-3); and
(h) a suppression of tumorigenicity 2 (ST2).

2. The cGVHD diagnostic biomarker panel of claim 1, wherein the panel comprises:
(a) a NK_{REG} Non-Cytolytic cell selected from one or more of: CD56^{Bright} CD355⁺ CD56; CD56^{Bright} Perforin^{Negative} CD56; and CD56^{Bright} Granzyme B^{Negative} CD56;
(b) a Cytolytic NK cell that is CD56^{Lo} Granzyme B^{Hi} CD56;
(c) a Naïve Helper T cell selected from one or more of: CD4+ CD45RA+ CD3; CCR7+ CD45RA+ CD4+; PD1− CD45RA+ CD4+; CD27+ CD45RA+ CD4+; and CD31+ CD45RA+ CD4+;
(d) a Memory T Helper Cell that is CCR7− CD45RA− CD4+;
(e) a Naïve Cytotoxic T Cell selected from one or more of: CCR7+ CD45RA+ CD8+; and CD27+ CD45RA+ CD8+;
(f) a Naïve Regulatory T Cell selected from one or more of: PD1− CD45RA+ Treg; and CD31+ CD45RA+ Treg;
(g) a chemokine selected from one or more of: CXCL9; CXCL10; and CXCL11;
(h) an intercellular adhesion molecule-1 (ICAM-1);
(i) a T-cell immunoglobulin and mucin domain-3 (TIM-3);
(j) a suppression of tumorigenicity 2 (ST2); and
(k) a matrix metalloproteinase-3 (MMP-3).

3. The cGVHD diagnostic biomarker panel of claim 1, or 2, further comprising one or more clinical factors selected from:
(i) malignant; or non-malignant disease;
(ii) recipient age;
(iii) graft type (PBSC, bone marrow, or umbilical cord);
(iv) donor gender; and recipient gender;
(v) HLA match; or HLA mismatch;
(vi) ABO match; or ABO mismatch;
(vii) sibling donor source; or unrelated donor source;
(viii) myeloablative; or reduced-intensity conditioning;
(ix) use of serotherapy;
(x) use of total body irradiation; and
(xi) days post-HSCT.

4. A diagnostic method of detecting the expression level of a biomarker panel in a patient receiving a hematopoietic stem cell transplantation (HSCT), the diagnostic method comprising:
(A) measuring, in a biological sample obtained from the patient, a relative cell % or a concentration of at least one of each type of biomarker in a diagnostic biomarker panel, wherein the diagnostic biomarker panel is set out in claim 1 or 2; and
(B) assigning a diagnosis of cGVHD or late acute GvHD.

5. The diagnostic method of claim 4, wherein the diagnostic panel comprises: (a) a NK_{REG} Non-Cytolytic cell selected from one or more of: CD56^{Bright} Perforin^{Negative} CD56; and CD56^{Bright} Granzyme B^{Negative} CD56; (b) a Naïve Helper T cell selected from one or more of: CCR7+ CD45RA+ CD4+; PD1− CD45RA+ CD4+; CD27+ CD45RA+ CD4+; and CD31+ CD45RA+ CD4+; (c) a Naïve Cytotoxic T Cell that is CCR7+ CD45RA+ CD8+; (d) a Naïve Regulatory T Cell selected from one or more of: PD1− CD45RA+ Treg; and CD45RA+ Treg; (e) a chemokine selected from one or more of: CXCL9; CXCL10; and CXCL11; (f an intercellular adhesion molecule-1 (ICAM-1); (g) a T-cell immunoglobulin and mucin domain-3 (TIM-3); and (h) a suppression of tumorigenicity 2 (ST2).

6. The diagnostic method of claim 4 or 5, wherein the patient is assigned a diagnosis of cGVHD where the biological sample has:
(a) decreased CD56^{Bright} CD355⁺ CD56; CD56^{Bright} Perforin^{Negative} CD56; or CD56^{Bright} Granzyme B^{Negative} CD56 as a % of total lymphocytes;
(b) increased CD56^{lo} G⁺ CD56 as a % of total lymphocytes;
(c) decreased CD4+CD45RA+ naïve helper T cells as a % of CD4+ selected from one or more of: CD4+CD45RA+CD3; CCR7+ CD45RA+ CD4+; PD1− CD45RA+ CD4+; CD27+ CD45RA+ CD4+; and CD31+ CD45RA+ CD4+;
(d) increased CCR7-ve CD45RA-ve CD4+ effector memory helper T cells as a % of CD4+ cells;
(e) decreased Naïve Cytotoxic T Cell selected from one or more of: CCR7⁺ CD45RA⁺ CD8; or CD27⁺ CD45RA⁺ CD8;
(f) decreased naïve regulatory T cells as a % of total regulatory T cells, selected from CD45RA+PD1-ve and CD45RA+CD31+;
(g) increased concentration of one or more of: CXCL9; CXCL10; and CXCL11;
(h) increased concentration of ICAM-1;
(i) increased concentration of ST2;
(j) increased concentration of TIM-3; and
(k) increased concentration of MMP3; or
(l) decreased CD56^{Bright} Perforin^{Negative} CD56; or CD56^{Bright} Granzyme B^{Negative} CD56 as a % of total lymphocytes;
(m) decreased CD4+CD45RA+ naïve helper T cells as a % of CD4+ selected from one or more of CCR7+ CD45RA+ CD4+; PD1− CD45RA+ CD4+; CD27+ CD45RA+ CD4+; and CD31+ CD45RA+ CD4+;
(n) decreased Naïve Cytotoxic T Cell that is CCR7⁺ CD45RA⁺ CD8;
(o) decreased naïve regulatory T cells as a % of total regulatory T cells, selected from CD45RA+PD1-ve and CD45RA+CD31+;
(p) increased concentration of one or more of: CXCL9; CXCL10; and CXCL11;
(q) increased concentration of ICAM-1;
(r) increased concentration of ST2; and
(s) increased concentration of TIM-3;
as compared to a control non-cGVHD or to a non-cGVHD standard at an equivalent time period post HSCT.

7. The diagnostic method of claim 6, wherein the patient is assigned a diagnosis of early-cGVHD, mid-cGVHD or late-cGVHD where there was: if the diagnostic markers were present at: less than 4 months for early-cGVHD; 4-8 months for mid-cGVHD; and greater than 8 months for late-cGVHD.

8. The diagnostic method of claim 7, wherein the patient is assigned a diagnosis of:
(a) early-cGVHD where the biological sample has:
(i) an increased concentration of one or more of: CXCL9; CXCL10; and CXCL11;
(ii) an increased concentration of ICAM-1; and
(iii) an increased enzyme activity of sCD13;
(b) mid-cGVHD where the biological sample has:
(i) an increased concentration of one or more of: CXCL9; CXCL10; and CXCL11; and
(ii) an increased concentration of ST2; or
(c) late-cGVHD where the biological sample has:
(i) an increased concentration of one or more of: CXCL9; CXCL10; and CXCL11;
(ii) an increased concentration of ICAM-1;
(iii) an increased enzyme activity of sCD13; and
(iv) an increased concentration of ST2;
as compared to a control non-cGVHD or to a non-cGVHD standard at an equivalent time period post HSCT.

9. The diagnostic method of claim 5, wherein the patient is assigned a diagnosis of moderate to severe cGVHD in the first year post-HSCT, where the biomarkers were as follows:
(a) decreased CD56^{Bright} CD3^{Negative} Granzyme B^{Negative} CD56 as a % of total lymphocytes;
(b) non-cytolytic NK cells no longer met criteria;
(c) increased concentration of one or more of: CXCL9; CXCL10; and CXCL11;
(d) increased concentration of ST2;
(e) increased concentration of ICAM-1; and
(f) increased enzyme activity of sCD13;
as compared to a control non-cGVHD or to a non-cGVHD standard at an equivalent time period post HSCT.

10. The diagnostic method of any one of claims 4-9, wherein biological sample is obtained from a pediatric patient.

11. The diagnostic method of any one of claims 4-9, wherein biological sample is obtained from an adult patient.

12. The diagnostic method of any one of claims 4-11, wherein biological sample is obtained from the patient at: +100 days from HSCT (±14 days); 6 months from HSCT (±1 month); and 12 months from HSCT (±1 month).

13. The diagnostic method of any one of claims 4-12, wherein biological sample is selected from the group consisting of: whole blood; plasma; and tissue.

14. The diagnostic method of any one of claims 4-12, wherein the detecting of the expression level of a biomarker is via one or more of the following assay methods: flow cytometry; microarray analysis; immunoassay; immunohistochemistry; enzymatic assay; and mass spectrometry.

15. The diagnostic method of claim 14, wherein immune assay is selected from: enzyme-linked immunoassay (ELISA); and meso scale.

16. The diagnostic method of any one of claims 4-15, wherein the cGVHD patient is preferentially chosen for administration of an immunosuppressive therapy (IST) selected from one or more of: sirolimus; mycophenolate mofetil; mycophenolate sodium; ibrutinib; ruxolitinib; belomosudil; thalidomide; azathioprine; pentostatin; daclizumab*; infliximab*; and rituximab*; or a biosimilar thereof*.

17. The diagnostic method of claim 16, wherein the cGVHD patient is further administered a steroid treatment.

18. A cGVHD risk assignment biomarker panel, the panel comprising:
(a) a CD19+lymphocyte;
(b) a transitional B cell selected from one or more of: a CD38− CD10− CD19+ B cell; a CD38^{dim} CD10^{lo} CD19+ B cell; and a CD38^{int} CD10^{int} CD19+B cell;
(c) an NK_{REG} Non-Cytolytic cell selected from one or more of: a CD56^{hi} CD355^{hi} CD56; a CD56^{hi} Perforin^{Negative} CD56; and a CD56^{hi} Granzyme B^{Negative} CD56;
(d) a Cytolytic NK cell selected from one or more of: a CD56^{dim} P^{hi} CD56; a CD56^{dim} CD69+CD56+; and a CD56^{lo} G^{hi} CD56;
(e) a CD3+ lymphocyte;
(f a PD1++CD45RA+ CD4 Follicular T cell;
(g) a PD1+ CD45RA+ CD8 T cell;
(h) a CD31− CD45RA+ CD4 T cell;
(i) an IgD+ CD27− CD19+ B cell;
(j) a lysophosphatidylcholine (LYSOC) selected from one or more of: LYSOC14:0; LYSOC16:1; and LYSOC20:3;
(k) a Taurine;
(l) an Aspartic acid;
(m) a Phenylalanine;
(n) an alpha-Ketoglutaric acid;
(o) a Fumaric acid; and
(p) a phosphatidylcholine (PC) PC40:6AA.

19. The cGVHD risk assignment biomarker panel of claim 18, wherein the panel comprises:
(a) a CD19+lymphocyte;
(b) a transitional B cell selected from one or more of: a CD38− CD10− CD19+B cell; a CD38^{dim} CD10^{lo} CD19+ B cell; and a CD38^{int} CD10^{int} CD19+ B cell;
(c) an NK_{REG} Non-Cytolytic cell selected from one or more of: a CD56^{hi} CD355^{hi} CD56; a CD56^{hi} Perforin^{Negative} CD56; and a CD56^{hi} Granzyme B^{Negative} CD56;
(d) a Cytolytic NK cell selected from one or more of: a CD56^{dim} P^{hi} CD56; a CD56^{dim} CD69+CD56+; and a CD56^{lo} G^{hi} CD56;
(e) a CD3+ lymphocyte;
(f) a PD1++CD45RA+ CD4 Follicular T cell;
(g) a PD1+ CD45RA+ CD8 T cell;
(h) a CD31− CD45RA+ CD4 T cell;
(i) a lysophosphatidylcholine (LYSOC) selected from one or more of: LYSOC16:1; and LYSOC20:3;
(j) a Taurine;
(k) an Aspartic acid;
(l) a Phenylalanine;
(m) an alpha-Ketoglutaric acid;
(n) a Fumaric acid; and
(o) a phosphatidylcholine (PC) PC40:6AA.

20. The cGVHD risk assignment biomarker panel of claim 18 or 19, further comprising one or more clinical factors selected from:
(i) malignant; or non-malignant disease;
(ii) recipient age;
(iii) graft type (PBSC, bone marrow, or umbilical cord);
(iv) donor gender; and recipient gender;
(v) HLA match; or HLA mismatch;
(vi) ABO match; or ABO mismatch;
(vii) sibling donor source; or unrelated donor source;
(viii) myeloablative; or reduced-intensity conditioning;
(ix) use of serotherapy;
(x) use of total body irradiation; and
(xi) days post-HSCT.

21. A risk assignment method of detecting the expression level of a biomarker panel in a patient about to receive a HSCT, the risk assignment method comprising:
(A) measuring, in a biological sample obtained from the patient, a relative cell % or a concentration of at least one of each type of biomarker in a risk assignment biomarker panel, wherein the risk assignment biomarker panel is set out in claim 17 or 18; and
(B) assigning a risk to the patient of developing cGVHD of either low, moderate or high;
as compared to a control non-cGVHD or to a non-cGVHD standard at an equivalent time period post HSCT.

22. The risk assignment method of claim 21, wherein the patient is assigned a diagnosis of cGVHD where the biological sample has:
(a) decreased CD56^{Bright} CD355⁺ CD56; CD56^{Bright} Perforin^{Negative} CD56; or CD56^{Bright} Granzyme B^{Negative} CD56 as a % of total lymphocytes;
(b) increased CD56^{lo} P⁺ CD56; CD56^{lo} CD69⁺ CD56; or CD56^{lo} G⁺ CD56 as a % of total lymphocytes;
(c) increased CD3+ T cells as a % of total lymphocytes;
(d) increased PD1++ CD45RA+CD4+ T cells as a % of total lymphocytes;
(e) decreased CD31− CD45RA+ CD4+ as a % of CD4+ cells;
(f increased PD1+ CD45RA+CD8+ T cells as a % of total lymphocytes;
(g) decreased CD19+ lymphocyte cells as a % of lymphocyte cells;
(h) decreased transitional B cell selected from one or more of: a CD38− CD10− CD19+ B cell; a CD38^{dim} CD10^{lo} CD19+ B cell; and a CD38^{int} CD10^{int} CD19+ B cell as a % of total regulatory B cells;
(i) decreased IgD+ CD27− CD19+ B cells as a % of total regulatory B cells;
(j) decreased concentration of taurine;
(k) increased concentration of aspartic acid;
(l) increased concentration of phenylalanine;
(m) increased concentration of alpha-ketoglutaric acid;
(n) increased concentration of fumaric acid;
(o) increased concentration of a lysophosphatidylcholine (LYSOC) selected from one or more of: LYSOC14:0; LYSOC16:1; and LYSOC20:3; and
(p) decreased concentration of phosphatidylcholine (PC) PC40:6AA;
as compared to a control non-cGVHD or to a non-cGVHD standard at an equivalent time period post HSCT.

23. The risk assignment method of claim 21, wherein the patient is assigned a diagnosis of cGVHD where the biological sample has:
(a) decreased CD56^{Bright} CD355⁺ CD56; CD56^{Bright} Perforin^{Negative} CD56; or CD56^{Bright} Granzyme B^{Negative} CD56 as a % of total lymphocytes;
(b) increased CD56^{lo} P⁺ CD56; CD56^{lo} CD69⁺ CD56; or CD56^{lo} G⁺ CD56 as a % of total lymphocytes;
(c) increased CD3+ T cells as a % of total lymphocytes;
(d) increased PD1++ CD45RA+CD4+ T cells as a % of total lymphocytes;
(e) decreased CD31− CD45RA+ CD4+ as a % of CD4+ cells;
(f) increased PD1+ CD45RA+ CD8+ T cells as a % of total lymphocytes;
(g) decreased CD19+ lymphocyte cells as a % of lymphocyte cells;
(h) decreased transitional B cell selected from one or more of: a CD38− CD10− CD19+ B cell; a CD38^{dim} CD10^{lo} CD19+ B cell; and a CD38^{int} CD10^{int} CD19+ B cell as a % of total regulatory B cells;
(i) decreased concentration of taurine;
(j) increased concentration of aspartic acid;
(k) increased concentration of phenylalanine;
(l) increased concentration of alpha-ketoglutaric acid;
(m) increased concentration of fumaric acid;
(n) increased concentration of a lysophosphatidylcholine (LYSOC) selected from one or more of: LYSOC16:1; and LYSOC20:3; and
(o) decreased concentration of phosphatidylcholine (PC) PC40:6AA;
as compared to a control non-cGVHD or to a non-cGVHD standard at an equivalent time period post HSCT.

24. The risk assignment method of any one of claims 21-23, wherein biological sample is obtained from a pediatric patient.

25. The risk assignment method of any one of claims 21-23, wherein biological sample is obtained from an adult patient.

26. The risk assignment method of any one of claims 21-25, wherein biological sample is obtained from the patient at one or more of: +100 days from HSCT (±14 days); 6 months from HSCT (±1 month); and 12 months from HSCT (±1 month).

27. The risk assignment method of any one of claims 21-26, wherein biological sample is selected from the group consisting of: whole blood; plasma; and tissue.

28. The risk assignment method of any one of claims 21-26, wherein the detecting of the expression level of a biomarker is via one or more of the following assay methods: flow cytometry; microarray analysis; immunoassay; immunohistochemistry; enzymatic assay; and mass spectrometry.

29. The risk assignment method of claim 28, wherein immune assay is selected from: enzyme-linked immunoassay (ELISA); and meso scale.

30. The risk assignment method of any one of claims 21-29, wherein the patient's cGVHD risk assignment is as follows:
(a) a high risk for a weighted sum of >2.1618;
(b) a low risk for a weighted sum of <−0.0890; and
(c) an intermediate risk for a weighted sum between −0.0890 and 2.1618.

31. The risk assignment method of claim 30, wherein a high risk patient preferentially chosen for early administration of an IST selected from one or more of: cyclosporine; voclosporin; tacrolimus; pimecrolimus; sirolimus; mycophenolate mofetil; mycophenolate sodium; ibrutinib; ruxolitinib; belomosudil; thalidomide; azathioprine; pentostatin; daclizumab; infliximab; and rituximab; or a biosimilar thereof.

32. The risk assignment method of claim 31, wherein the high risk patient is further administered a steroid treatment.

33. The risk assignment method of claim 32, wherein the steroid treatment is selected from one or more of: prednisone; methylprednisolone; dexamethasone; beclomethasone; and budesonide.

34. The risk assignment method of claim 30, wherein a low risk patient is preferentially chosen for reduced duration of IST prophylaxis or early discontinuation of IST.
